SEQUENCE LISTING (12 sequences)

SEQ ID NO: 1 - DNA, 15631 bp, Homo sapiens
  source 1..15631: mol_type = genomic DNA
acatttgcgggaacgcagagcggagcgtggagagcggagcgaagctggataacagggtaaagggagtgggtggggggcctctgtccagggagggcggctcgggtcgaggaccagggtccggactgggagcaatgacggggagcctgcctgacctctcgctggccacaactatgccctgcaccttgcgtgggatgtgcgccacgtctgtctctgtaattccccctggcaggtctgggacccccctgagactccgtcagaagattcggtcctccccataggtggctttacctctaagcatcccgccctcttttcccccatcgcccgccctttttgagcccgaaagcttgcagccaacttccaactccctgtcctgtcctaggtaacccctccaccccgccattctcctatcccgtgtctgtccccatccctgtgacccctgacccctggcctttgccactccccagggaccgatgatgtggcgaccatcagttctgctgcttctgttgctactgaggcacggggcccaggggaagccatccccagacgcaggccctcatggccaggggagggtgcaccaggcggcccccctgagcgacgctccccatgatgacgcccacgggaacttccagtacgaccatgaggctttcctgggacgggaagtggccaaggaattcgaccaactcaccccagaggaaagccaggcccgtctggggtaagagagacattcggttggggcgtgtccagaggtggggcttaggagagagacgcgggggtatcgacaggggtcagagaaatggcgtggactgaacttcatttctttgtgagaaatgaaattctaccttctatccttgggaaaaagaaattaagttaaaattgaggcctaaagaggacctttgagaccgggcgtggtggctcatgcctgtaatcccagcactttgggaggccaaggcaggccgatcacttgaggtcaggagttcgagaacagcctggccaacatggtgaaatcccgtctctactaaaaatacaaaattaggcaggcgtggtggtgggcacctgtaatcccagctactcgggaggctgaggcaggagaatcgcttgaacctgggaggtggaggttgcagtgagccaaaattgcaccactgccctccagtcagggcgacaaagtgagactccgtctaaaaaacaacaacaacaataaaaaagaggacctttgagggcagcaaaatctgggttagattctgccccctaagcgctccttattcttttgtcccagacatctcccctccccaagcctcagtttcctgctctgtaaatggcagggactggtgtggaaaatgaggtttggcctttaaggagcccggccagtgcaggagagtggcagagccctgagtttcagggtgagctgggtggaatggagccctgtccaccctctccgtccaggcctgagcgcaggggggcgcgatgggagcagccgccttcttctgtgcatcctcctgacactgattgagcctgcaaaggtttatcgagcacctgctatgtgccaggcactgctctaggtgctgaagtcacaacagctaacacatctgtcctcctgaagtttacactgtggtgagggaaacagcaagaagtcaataagtagaaagtggatcaaatagagataaatgcatccaagaaagataaagcaagagagtcagaaagtgtggaggggagaggttgttggtgggggggagttgcaatttttatttatttatttatttatttatttacttattgagatggagtctcgctctgtcacccaggctggagtgcagtggtgcgatctcggctcactgcaacctccacctcctgggctcaagcaattgtcctgtctcagcctcccaagtagctgggattacaggcgctagccaccacgcccagctaattttgtatttttattttttatttttatttttgttgttgttgtttttgagatggagtctcgctctgtcacccaggctggagtgcagtgacgtgatctcggctcagtggaacctccgcctcccgggttcaaccagttgttctgcctcgtactccctagtagctagagtcctagctacaagtgaacgctacaagtcctagctacaagtgaacgcctccacatccagcaaagttttttttctttccttttttttttttggagacagagtgagactctgtctcccgggctagagtgcagtggcgtgatctcagctcatgcaacctctgcctcccaggttcaagcaattctcctgcctcagccttccgagtagctgggactatgggtgcacgcccacacgcccagctaatttttttttttttttttgtattttagtagagatggggtttcactgtgttgcccaggctggtctcgagctcctgagctcaggcaatccgcccgccttggcctcccaaagtgctggaattaatgcgtgagccacagcacccagcccaggcccaggtaatttttcttttttttcttttttttttttttttgagacggagttttgctcttgttgcccaggctggagtgcagtggcgcaatctcggctcaccacaacctccgcctcctgggttcaagtgattctcctgcctcagccttccgagtagctgggattacaggcatgcaccaccatgcccggctaattttgtatttttagtagagatggggtttctccatgttggtcaggctggtatcgacctcctgacctcaggtgatctgcccacctcggcctcccgaagtgctgggattataggcatgagccaccgcacccggctggagctgcaattttaaacagggaacttggacaaggcctccatgagaaggtggtatgtggtatttgagccatgtgggtatcggagtgaatagcattccaggcagacagaacagttaagtgcaaaggccctgaggcaggagtgggcttggagtgtcagagcaacagttctgaggcccgtgtggctggaactcagtgagaaaggggagagagtgggaggtgagggcagagaaggagcaaaggggttcagcatggtggttcatgtgtgtgatcccagcactttgggaggccaaggctgggggatcacttgagcccaggagttcaagaccagcctgggcaacatagtgataccccgcctctacaaaacaaatacaaaaattagcccagcatggtggcgtgctcctgtagtcccagatactagggaggctgaggagggaggatcacttgggcctgggaggtcaaggctgcagtgagccaaaatcatgccgctgcactccagcctaagcaacagagcaagactttgtctcaaaaagagagagggccaggtgcagtggctcacgcctgcaatcccagcactttgggagtccaaagcgggcggattgcctgagaccatgcaaagggggtcagatcatgcaatgcaaagccttttaggccacgatgaagatttaggcttttctccaattgagagtagaactgagggagggcatgagctgacttaggttttgagagaatcctgctggctgcgaggtggggaacagactgtagggagcaagaagcagggagcccagtgaggaagcgactgcgatagtccaggtggaagacaagacaagatggtggtcagatcagggtggagacagtggcagtggtccccaggactgctggctgggagattgtgcttggatgggcatgcatgaaggagaaatggaggaagaagggcagtgacaggttagcacatttagcttagcactaatggcaatgccaggcattgtcctaagtgctttattattgttattattattattattttgagacgcagtctcgctctgtctcccagtctggagtgcagtggcgtgatctcggctcactgcaagctccacctcccgggttcatgccattctcctgcctcagcctcccgagtagctgggactacaggcgcccaccaccacgcccagctaatttttttgtatttttagtagagacggggtttcgccgtgttagccaggatggtctagatctgctgacctcgtgatccacccacctcggcctcccaaagcactgggattacaggcgtgagccatggcgcctggcctttttttttttttttttttttttgagacagagtctcactctgtcatccaggctggagtgcaacggcgcgatctcggctcactgcaagctgcgcctcccaggttcacgccattctcctgcctcagcctcccgagtagctgggactacaggcacctgccaccacgcccggctaattttttgtatttttagtagagacggggtttcaccatgttagccaggatggtctcgatctcctgaccttgtgatccgccctcctcggcctccaaagtgttttttgtttgtttgtttgtttttgagacaaggtcttactctgttgcccaggctggagtgcagtggcacgatctcggctcactgcaacctacgcctcctgggttcaagcaattctcctgcctcagcctcccgagtagtggatattacaggcatccaccactgtgcccggctactttttgtatttttagtagagacggtttcaccatgttggccaggctggtctggaactcctcacctcaggtgatctgcccacttcggcctcccaaagtgctgggattacaggcgtgagccaccgtgcccggcctattattgttattattatttgagacggagtctcgctctgttgcccaggctggagtgcagtggtactatctcggctcactgcaacctccacctcccagattcaaccaattctcccgcgtcagcctccagagtagctgggatttcaggtgcgtgccaccatgccctgctaatttttgtatttttagtagagacaaggtttcaccatgtggaccaggctggtctggaacgtctgatctcaagtgattcgcctgtcttggcttcccaaagtgctgggattacaggcgtgagccaccgcgcctgtcctgtcctaagtgctttacatggcctaactcatttattccccacaaccgcccttttggggtaggtaccgttttttcttcctccgttttacagaggggtaactgagacacagagaggttaagtcacttgcccacagtcacacagccagtaagaggaagtggctggatttggacctaaaatgtctggctgtggagcccaagcgcttaacaacaccaggaacctcagagaagggtgtgattccggggacagagtggggccctagacagactggagcagagaccgcctggtgcgggaggtggggcggtgactcaggcaggcctggagagggctctgctgtctcggattttcgactttagggagggggccgcgggggcctagggacggtgacagggaggagggctccaggtagtctctgttaattagagattgggagaggaccggcctggcaaggcgtcaccaagggagaggagaggggcccgagagctgataggcgtggcccaagagaaggggagagatggaacctaaagtggcttgacatgcgacagggaggggcacaggagcccggaggagcatggggaggggcagagggagactgagcccccggggttgcgggtgatggtcctcaggcagtgcctgattcagccgggagcaggcaggggaaagccacggggctcagcacagggcagggctggagggagaggagcctgaaattgacacgcgcagcctggtaagggggaggggcatggggcgggtcggggcctgagacagcgggggatcgggggtgtggccgtggctgtctgggttcagagtcaatcttgcaacagggcccccttggcaagggtcacagtgcgacgccgcgctccccttcaccaccccagctcggcccagcgagaggtgcagcacgtgcaggccgagggcggggccggcgcggagagggtggccctgcatcgtagtaggtccctcccccgcagtgctcccagcaggcccgctcgttctggagaagggcgtgactcgacctgggggtggaccctaggctgaggggtagggtcctccggtgggcttggtcccgaaattgacccgcgcccctccccagttagcccgcggaccgtggactgtattttcagccccggatccgaagtgtcccagggggcgtggcccgtgcgaggggcggggcctgggggcgggaccagagcctgacgtgtgcccgccccggcttctaggcggatcgtggaccgcatggaccgcgcgggggacggcgacggctgggtgtcgctggccgagcttcgcgcgtggatcgcgcacacgcagcagcggcacatacgggactcggtgagcgcggcctgggacacgtacgacacggaccgcgacgggcgtgtgggttgggaggagctgcgcaacgccacctatggccactacgcgcccggtacgcggcgagcccccgaccctgctccccatacaccgtgaccctgaccttctggaccgcctcattctgagaaccctgaacccatttacccggagtccctggcccctaaccaggctctacccacttttaggagcccataactgtgagatcttcagtttctcacctctggcttctcttttttgtttttcgtctgcttatttttctggtatttagctgcatgctaaaaacgatctttttttcaaacatgaagtcacaattaggtgctaaaagccactgaacgcacccaaacagggacttgaaacctgggccctcaaattaaaagtccaatgctctaccaactaagctacccaggttcccttcagggctttcattctgggactcaccacctctgtgacaatatgactagggtacttgagatccctggccaaggacacctactttccatgccttgtcatcctgacccctaacttctgtgacctctcatcctgagaaccccaaagccagttactcagcccctgactctcagatccctcctttctgtcccaagtcccagcctcagtgccccacaccctgccacacacacagccctcattctctccctcctctcctctctgtccctggccccagcctccccgtccagtctgtcctccacagggctccgggggcggggttcttcatgcccagcactgactctgcccctccctccccctcctgggcccccaggagacggtcccactccccagccgggacgtttcctcctgtggctttgtttttctgcattttctgcaccaaatggattttctgtggttttcctgaactcaccacactcttcaaacctctaccttctgcccatattgttccctccagttacaatgcccgtcccctcctctcagtcaagccatctcagtccctgtaagctaccccatcacagccttagtctggggccaaaaggcccactgtcaactcatagtgggccattcctgtctccacacgcttggatcagttatttcttctctctgagaatcaatttttgcattcagaaagtagaatacatggaaaaacccactgtatgtgcttttttgtgatgattaaatgcaatattgtgcataaagagcttagcatataatctctcacaagttagctaatttattgggtggatataatatgttacatagcctgggtgccatggctcacgcctgtaatcccagcactttgggaggccgaggtgggtggatcacgtgagatcagaagttagagaccagcctggccaatgtggcaaaaccccgtcttaccacaaaaaaatacttagctgggcgtgatggtgtgcacctgtgatcacagctactctggagggtgaggcaagagaatcacttgaacccaggaggcagaagttgcagtgagccgagatcacatcactgcactctggccttggcgacagagtgagactctgtctcaaaaaaaaaaaaatatatatatatatagatagatagatagatagatagatatagatatacacacacatactaataatatgttatataacatattatatacttaaatatatatacaaattatatataaagtatcaatatatgcttttttttttttgtgagggagttttgctcttgttgcccaggctggagtgcagtggcgtggtctcggctcactgcaacctccgcctcccgggttcaagcgattttcctgcctcagcctcccaagtagctgggattacaggcatgcgccaccacgcctggctaattttgtatttttagtagagatggggtttctccatgttggtcaggctggtctcgaactcccaacctcaggtgatctgcccacctcggcctcccaaagtgctgtgattacaggtttgagccaccgcacccggcctacttttttttttttttttttttttttgagatggaatggagtttcactcttgttggccaggctgcagtgcaatggcaaggtctcggcttactacaacctcatcctcctgggttcaagcaattctcctgcctcagcctcacgagtagctaggactacaggcgcccaccaccatgccaggcaaatttttgtatttttagtagagaaggagtttcaccatgttgaccaggctggtctcgaactcctgacctcaggcgatccacccacctcggcctcccaaagtgctgggattacaggtgtgagccactgtgccaggcttctaaaatttttttttttttttttttgagacatagcctccctctgtcgcccaggctggaaagcagtggtgcagtctcagctcacggcaacctccgcttcctgggttcaatcgactcttgtgcctcagactcccgagtagctgggattacaggcgtgccccaccatgcccggctaagtatttttttggtagagatggggtttcgccatgttggccaggctggtctcaaactcctgacctcaagtgatctgccagcttcagcctcccaaagtgctgctattacaggcgtgagccactgcatccagcctactgatatatacttattaactccacagaaatctttgtaccccagtaggatttactttgtctttgttttaacctgcttggcgtttcctccattaaagctcatctcactgagacctgtccttccccaggtgaagaatttcatgacgtggaggatgcagagacctacaaaaagatgctggctcgggacgagcggcgtttccgggtggccgaccaggatggggactcgatggccactcgagaggagctgacagccttcctgcaccccgaggagttccctcacatgcgggacatcgtgattgctgtgagtggcggctggggaaccctgtcccccacacccttccggggacccaggcttccggttcaggtcctgcttcccagcaccgacctgggggcaggcagtcacctggttctccagcatcttgccggggaagccaggccgcaaacacaggtcagaggacggtgacctttcccacctccactctcagtcccctggggtctgatctctcctcgtctggccttcccctccctgtgctcgcatttggggtccaggcccccagccttccctcagacagccactgtgccccaccttatacaatttcttttctttcttttttttttttgagacatagtctccctcggttgcccaggctggaaagcagtggtgcgatctcagctcactgcaacctccgcctcccgggttcaagcaattctcctgcctcagcgtcccgagtagctgggattacaggggcgcaccaccacacctggctaatttttagtagagacagggtttcttcatgttggttaggctggtctcaaacttttgacctcaggtgatccgcccgcctcagcctcccaaagtgctaggattataggcatgagccaccgtgcccggccaatttttaatttttttttctagtagagatgggatttcaccatgttggccaggctggtcttgaactccggacctcaggtgatctgcccaccttggcctcccaaagtgctgggattacaggcatgagccaccgtgcctggccccaaaatgtgctttgttctatagaagaaggctttttttttctttccttcctcttttttttttttagtttttgagacagggcctaactctgttacccagaccggagggcagtggctcaatctcagctcactgcaacctcaaactcttgggctcaagtaatcctcccacctccgccttctgagaagctgaggccaccaggcacaagccaccatgtctggctaatgtttttttttttttttttttcagacggagtctcgctctgtcgcccatgctggagtgcagtgatgcgatctcggctcactgcaagctctgcctcccgggttcactccattcttctgcctcagcctcccgagtggctgggactacaggcgcatgccgccatgcccggctaatttttttttttttttttttagtagagacggggtttcactgtgttagccaggatggtctcgatctcctgacctcgtgatccacccacctcggccttccaaagtgctgggattacagttgtgagccaccacgcccggccatgcctggctaatttttaaattttgtgtagagacagggtctcgccatgttgcccaggctagtctcaaaatcctgggctcaatcgatcctcccgcctcggcctcccaaagtgctgggattacaggtgtgagccaccgtgcccagcccacagtcttatagtgaatgagtttggatataaattggggaacaaaagtcgttttgctccagaagggcacaaaaacaaggagcggaggtgtccagtgtgtcagaagaagccccaaagtgcaccatgttcacacataaagcagaggtaggttctaggctctgctaaacataaaggagcttctccccttggagggtgtttgctggggcttgagcaagttattagggacccaggacagctccccttgtgagcaacaaacctgtgcactgctggacattagggcctctgggctctggccccgtaaatgcaggtggtgcctgtttgatgataacatcccagaaagcccgcaaatttcccacatgccacctgttgagactgctctactgcctgcctcccacagctgcccggagctaaatttccaagagaacctctcactcttaccccaggggtccagcctcccccaggagccagggtcatcggcccccagcctcaatgcccctttctcctccaggaaaccctggaggacctggacagaaacaaagatggctatgtccaggtggaggagtacatcggtgagtgggccccaatttcttcttgggatgcctgtcctctctcaggcatgggcagggttggtgggggtagccggaggtacatcacccatcatcagagaacagtggccctcagacatgggggcaggggcactcactgcttgagttcatttagccacccaacatttattgagcacctactctataccagacacaaaaagcatctattgagcacctgctgtgtgtcacgcccaggatacacttactgagcacctactgcatatgggtggttgcgcctacagcagacaataacacagacaaatcccttcttgggggtctggagtttctacaaaatcaaggctcaggggctatgtttagttgagaatgggggtcaggaagctggtcttgaacctgcgcctgcacagcaaacactgtttcctgttacatgggatgttgggggatcctcacaataccccctgagttctgagttctccctgggcccctgccactgtccctgcccctagcgggcaaaaaatttagtcaaggagaacgctaacaaggaattttttttttttttttttttttttgagacagtttttttctgttgctcaggctggagcgcggtggcgtggtatcggctctctgcaacctccgcctcccgggctcaagagattcttctgcctcagcctcctgagtagctgggattactggtgcctgccaccacgcctggctaatttttgtatttttagtggagatgggatttcaccatgttggccaggctggtctagaactcccaacctcaggtgatctgctcatcttggcctcccaaagtggtgggattacaggcgtgagcaaccacgcccggccaggaatttttttttaagtttgaaaacattgtttattcttttatttttttctgaccacatcctacatgagaaaacatttttctttctttccttctctcactttctttctttctgtcactctttctttctatctgtctgtctgtgtgtgtgtgtgtctctctctttattaaatagagatgggttctcactatattgtccaggctggtctagaactcctgggctcaagcaatcttcccaccttggcctcccaaagagctgagattacaggcatgaatcaccacacctggctgagagaacatttttatatttttttcttaagagatggggttgggccaggcgcagtggctcatacctgtaatcccagcactttgggaggctgagatggatggatcatgaagtcaggagttcgagaccagcctggccaagatggtgtactaaaaatacaaaaattagccgggcctgttggcaggagcctgtaatcccagttactctggggactgaggcaagagaatctctggaacccgggaggcaaaggttgcagtgagctgtaatcgcgccattgcacttcagtctgggcaacaagagcgaaactccatcttaaaaaaaaaaaaaaaagatggggtcttgccctgtcgcccaggctggagtgcagtggggtgatcttggctcactgcagccttgacctcctgggctcaagggatcctcccacctcagcctcccaagtagctgagactccaggcacagtgcggcctctggagatacttctggttgtcacttctagaggggtgaggatgctactgacgtctcctggatagagcccagagatgctccttagcattctccggtgcacaggacaggccctgacaacaaggaggtcacaggccccagatgccagcagctgtccccatctctttttttttttttttgagacagagtctcgctattgtcgcccaggctggagtgcagtggcatgatctcggctcactgcaacctccgcctcccaggttcaagcaattctcttgtttcagcctcctgagtaggtgggattatgggcgtgcactaccacgcctggctaattttgtatttttagtagagacggggtttcaccattttggtcaggctggtcttgaactcttgacctcaggttatccacctgcctcggcctcccaaagtgctgggattacaggcgcgaaccaccgcacccagcctatcttctcaattacttcatcccattaagacttcatcttccattcctcagctctaccaggcagacactgtcactatcctccattatatagatgacacctgaggcccaggggggttgaagggatctgctgaagacaatgggaagaggcagggctaggatttgaaccttggtgtttgggctctggcacctatttcttaactcctccccaaccagtaacatatcaaaatgtataaaattggggtgggtgccatggctcacgcctgtattcccagcactttaggaggccaaggtgggtggatcacttgaggtcaagagttcaagaccagcctggccaacatgatgaaaccctgtctctactaaaactagaaaaattagctgggcgtggtggcgggtacctgtaatcccagctacttgggaggctgaggcaggagaatcacttgaacccagaaggtgcaggttgcagtgagctgagattgtgtcactgcactccagcctgtaatcccagcactttgggagactgaggcaggtggatcatggggtcaggagtttgagagcagcctggccaagatggtgaaaccccgcctctactaaaaatataaaaattagctgggtgtggtggcacgcacctgtaatcccagctacttgggaggctgaggcaggagaatcgcttgaacctgggaggcagaggttgcagtgagctgagattgcgccattgcactacagcctgggcaacaagagcgaaactccatcttaaaaaaaaaaagggggtggggtcttgccctgttgcccaggctggagtgcagtggggtaatcacagcgagactccatctcaaaaaaaaaaaaaaaaatttgtagaaacagggtcttgctatgttgcccaggttggtcttgaatcttggcctcaaatgaccctcctgcctgggtctcccaaagtgttggattacagtcatcactgcacctggcatctctgtcacctgctaactggccagaaacatcctcgcacgttcctcctgcatagacattgcttactatgtgccaggcactgccttgcccattgcacacactacttcacgtgtgtgtcaatgaaatcaatcaatcatgcaatcaaccaatcaaatgaatttcacctgtaacacaaatgattgcttataagtcaggttttgttactaattccatcgaaacagaagaggacgctgaggcgagagagagggtagtgagttgctcaagggcctggttcaggaagtatcagagccaggatcagcccccagctccactagacccccagagctgcccctgaccttgtcccctctgtcccggcccccagcggatctgtactcagccgagcctggggaggaggagccggcgtgggtgcagacggagaggcagcagttccgggacttccgggatctgaacaaggatgggcacctggatgggagtgaggtgggccactgggtgctgccccctgcccaggaccagcccctggtggaagccaaccacctgctgcacgagagcgacacggacaaggtgcagtgacggggcctcggcaggagcgaggagcgggtgggcattgcgggccagactccagaaaggagcaagacctgggcctggagctcagggtcccttttaggtgggataaaaaaagagggacagagagagggaggaaaagagagggcacggagccccagaaagagagggggacagagacccagagacccaaagagagaaggacagggaccaagacggggacagattcagagagaaagggacagaggcccagagaacaagggtcccagagacttcgggacacgcttggatgcagggagggcttttgaaagcagggccgtgttgtcccctctgaaccctgaccctccctccaggatgggcggctgagcaaagcggaaatcctgggtaattggaacatgtttgtgggcagtcaggccaccaactatggcgaggacctgacccggcaccacgatgagctgtgagcaccgcgcacctgccacagcctcagaggcccgcacaatgaccggaggaggggccgctgtggtctggccccctccctgtccaggccccgcaggaggcagatgcagtcccaggcatcctcctgcccctgggctctcagggaccccctgggtcggcttctgtccctgtcacacccccaaccccagggaggggctgtcatagtcccagaggataagcaatacctatttctgactgagtctcccagcccagacccagggacccttggccccaagctcagctctaagaaccgccccaacccctccagctccaaatctgagcctccaccacatagactgaaactcccctggccccagccctctcctgcctggcctggcctgggacacctcctctctgccaggaggcaataaaagccagcgccggga

SEQ ID NO: 2 - DNA, 15631 bp, Homo sapiens
  misc_feature 13482: note = n is a, c, g, or t
  source 1..15631: mol_type = genomic DNA
acatttgcgggaacgcagagcggagcgtggagagcggagcgaagctggataacagggtaaagggagtgggtggggggcctctgtccagggagggcggctcgggtcgaggaccagggtccggactgggagcaatgacggggagcctgcctgacctctcgctggccacaactatgccctgcaccttgcgtgggatgtgcgccacgtctgtctctgtaattccccctggcaggtctgggacccccctgagactccgtcagaagattcggtcctccccataggtggctttacctctaagcatcccgccctcttttcccccatcgcccgccctttttgagcccgaaagcttgcagccaacttccaactccctgtcctgtcctaggtaacccctccaccccgccattctcctatcccgtgtctgtccccatccctgtgacccctgacccctggcctttgccactccccagggaccgatgatgtggcgaccatcagttctgctgcttctgttgctactgaggcacggggcccaggggaagccatccccagacgcaggccctcatggccaggggagggtgcaccaggcggcccccctgagcgacgctccccatgatgacgcccacgggaacttccagtacgaccatgaggctttcctgggacgggaagtggccaaggaattcgaccaactcaccccagaggaaagccaggcccgtctggggtaagagagacattcggttggggcgtgtccagaggtggggcttaggagagagacgcgggggtatcgacaggggtcagagaaatggcgtggactgaacttcatttctttgtgagaaatgaaattctaccttctatccttgggaaaaagaaattaagttaaaattgaggcctaaagaggacctttgagaccgggcgtggtggctcatgcctgtaatcccagcactttgggaggccaaggcaggccgatcacttgaggtcaggagttcgagaacagcctggccaacatggtgaaatcccgtctctactaaaaatacaaaattaggcaggcgtggtggtgggcacctgtaatcccagctactcgggaggctgaggcaggagaatcgcttgaacctgggaggtggaggttgcagtgagccaaaattgcaccactgccctccagtcagggcgacaaagtgagactccgtctaaaaaacaacaacaacaataaaaaagaggacctttgagggcagcaaaatctgggttagattctgccccctaagcgctccttattcttttgtcccagacatctcccctccccaagcctcagtttcctgctctgtaaatggcagggactggtgtggaaaatgaggtttggcctttaaggagcccggccagtgcaggagagtggcagagccctgagtttcagggtgagctgggtggaatggagccctgtccaccctctccgtccaggcctgagcgcaggggggcgcgatgggagcagccgccttcttctgtgcatcctcctgacactgattgagcctgcaaaggtttatcgagcacctgctatgtgccaggcactgctctaggtgctgaagtcacaacagctaacacatctgtcctcctgaagtttacactgtggtgagggaaacagcaagaagtcaataagtagaaagtggatcaaatagagataaatgcatccaagaaagataaagcaagagagtcagaaagtgtggaggggagaggttgttggtgggggggagttgcaatttttatttatttatttatttatttatttacttattgagatggagtctcgctctgtcacccaggctggagtgcagtggtgcgatctcggctcactgcaacctccacctcctgggctcaagcaattgtcctgtctcagcctcccaagtagctgggattacaggcgctagccaccacgcccagctaattttgtatttttattttttatttttatttttgttgttgttgtttttgagatggagtctcgctctgtcacccaggctggagtgcagtgacgtgatctcggctcagtggaacctccgcctcccgggttcaaccagttgttctgcctcgtactccctagtagctagagtcctagctacaagtgaacgctacaagtcctagctacaagtgaacgcctccacatccagcaaagttttttttctttccttttttttttttggagacagagtgagactctgtctcccgggctagagtgcagtggcgtgatctcagctcatgcaacctctgcctcccaggttcaagcaattctcctgcctcagccttccgagtagctgggactatgggtgcacgcccacacgcccagctaatttttttttttttttttgtattttagtagagatggggtttcactgtgttgcccaggctggtctcgagctcctgagctcaggcaatccgcccgccttggcctcccaaagtgctggaattaatgcgtgagccacagcacccagcccaggcccaggtaatttttcttttttttcttttttttttttttttgagacggagttttgctcttgttgcccaggctggagtgcagtggcgcaatctcggctcaccacaacctccgcctcctgggttcaagtgattctcctgcctcagccttccgagtagctgggattacaggcatgcaccaccatgcccggctaattttgtatttttagtagagatggggtttctccatgttggtcaggctggtatcgacctcctgacctcaggtgatctgcccacctcggcctcccgaagtgctgggattataggcatgagccaccgcacccggctggagctgcaattttaaacagggaacttggacaaggcctccatgagaaggtggtatgtggtatttgagccatgtgggtatcggagtgaatagcattccaggcagacagaacagttaagtgcaaaggccctgaggcaggagtgggcttggagtgtcagagcaacagttctgaggcccgtgtggctggaactcagtgagaaaggggagagagtgggaggtgagggcagagaaggagcaaaggggttcagcatggtggttcatgtgtgtgatcccagcactttgggaggccaaggctgggggatcacttgagcccaggagttcaagaccagcctgggcaacatagtgataccccgcctctacaaaacaaatacaaaaattagcccagcatggtggcgtgctcctgtagtcccagatactagggaggctgaggagggaggatcacttgggcctgggaggtcaaggctgcagtgagccaaaatcatgccgctgcactccagcctaagcaacagagcaagactttgtctcaaaaagagagagggccaggtgcagtggctcacgcctgcaatcccagcactttgggagtccaaagcgggcggattgcctgagaccatgcaaagggggtcagatcatgcaatgcaaagccttttaggccacgatgaagatttaggcttttctccaattgagagtagaactgagggagggcatgagctgacttaggttttgagagaatcctgctggctgcgaggtggggaacagactgtagggagcaagaagcagggagcccagtgaggaagcgactgcgatagtccaggtggaagacaagacaagatggtggtcagatcagggtggagacagtggcagtggtccccaggactgctggctgggagattgtgcttggatgggcatgcatgaaggagaaatggaggaagaagggcagtgacaggttagcacatttagcttagcactaatggcaatgccaggcattgtcctaagtgctttattattgttattattattattattttgagacgcagtctcgctctgtctcccagtctggagtgcagtggcgtgatctcggctcactgcaagctccacctcccgggttcatgccattctcctgcctcagcctcccgagtagctgggactacaggcgcccaccaccacgcccagctaatttttttgtatttttagtagagacggggtttcgccgtgttagccaggatggtctagatctgctgacctcgtgatccacccacctcggcctcccaaagcactgggattacaggcgtgagccatggcgcctggcctttttttttttttttttttttttgagacagagtctcactctgtcatccaggctggagtgcaacggcgcgatctcggctcactgcaagctgcgcctcccaggttcacgccattctcctgcctcagcctcccgagtagctgggactacaggcacctgccaccacgcccggctaattttttgtatttttagtagagacggggtttcaccatgttagccaggatggtctcgatctcctgaccttgtgatccgccctcctcggcctccaaagtgttttttgtttgtttgtttgtttttgagacaaggtcttactctgttgcccaggctggagtgcagtggcacgatctcggctcactgcaacctacgcctcctgggttcaagcaattctcctgcctcagcctcccgagtagtggatattacaggcatccaccactgtgcccggctactttttgtatttttagtagagacggtttcaccatgttggccaggctggtctggaactcctcacctcaggtgatctgcccacttcggcctcccaaagtgctgggattacaggcgtgagccaccgtgcccggcctattattgttattattatttgagacggagtctcgctctgttgcccaggctggagtgcagtggtactatctcggctcactgcaacctccacctcccagattcaaccaattctcccgcgtcagcctccagagtagctgggatttcaggtgcgtgccaccatgccctgctaatttttgtatttttagtagagacaaggtttcaccatgtggaccaggctggtctggaacgtctgatctcaagtgattcgcctgtcttggcttcccaaagtgctgggattacaggcgtgagccaccgcgcctgtcctgtcctaagtgctttacatggcctaactcatttattccccacaaccgcccttttggggtaggtaccgttttttcttcctccgttttacagaggggtaactgagacacagagaggttaagtcacttgcccacagtcacacagccagtaagaggaagtggctggatttggacctaaaatgtctggctgtggagcccaagcgcttaacaacaccaggaacctcagagaagggtgtgattccggggacagagtggggccctagacagactggagcagagaccgcctggtgcgggaggtggggcggtgactcaggcaggcctggagagggctctgctgtctcggattttcgactttagggagggggccgcgggggcctagggacggtgacagggaggagggctccaggtagtctctgttaattagagattgggagaggaccggcctggcaaggcgtcaccaagggagaggagaggggcccgagagctgataggcgtggcccaagagaaggggagagatggaacctaaagtggcttgacatgcgacagggaggggcacaggagcccggaggagcatggggaggggcagagggagactgagcccccggggttgcgggtgatggtcctcaggcagtgcctgattcagccgggagcaggcaggggaaagccacggggctcagcacagggcagggctggagggagaggagcctgaaattgacacgcgcagcctggtaagggggaggggcatggggcgggtcggggcctgagacagcgggggatcgggggtgtggccgtggctgtctgggttcagagtcaatcttgcaacagggcccccttggcaagggtcacagtgcgacgccgcgctccccttcaccaccccagctcggcccagcgagaggtgcagcacgtgcaggccgagggcggggccggcgcggagagggtggccctgcatcgtagtaggtccctcccccgcagtgctcccagcaggcccgctcgttctggagaagggcgtgactcgacctgggggtggaccctaggctgaggggtagggtcctccggtgggcttggtcccgaaattgacccgcgcccctccccagttagcccgcggaccgtggactgtattttcagccccggatccgaagtgtcccagggggcgtggcccgtgcgaggggcggggcctgggggcgggaccagagcctgacgtgtgcccgccccggcttctaggcggatcgtggaccgcatggaccgcgcgggggacggcgacggctgggtgtcgctggccgagcttcgcgcgtggatcgcgcacacgcagcagcggcacatacgggactcggtgagcgcggcctgggacacgtacgacacggaccgcgacgggcgtgtgggttgggaggagctgcgcaacgccacctatggccactacgcgcccggtacgcggcgagcccccgaccctgctccccatacaccgtgaccctgaccttctggaccgcctcattctgagaaccctgaacccatttacccggagtccctggcccctaaccaggctctacccacttttaggagcccataactgtgagatcttcagtttctcacctctggcttctcttttttgtttttcgtctgcttatttttctggtatttagctgcatgctaaaaacgatctttttttcaaacatgaagtcacaattaggtgctaaaagccactgaacgcacccaaacagggacttgaaacctgggccctcaaattaaaagtccaatgctctaccaactaagctacccaggttcccttcagggctttcattctgggactcaccacctctgtgacaatatgactagggtacttgagatccctggccaaggacacctactttccatgccttgtcatcctgacccctaacttctgtgacctctcatcctgagaaccccaaagccagttactcagcccctgactctcagatccctcctttctgtcccaagtcccagcctcagtgccccacaccctgccacacacacagccctcattctctccctcctctcctctctgtccctggccccagcctccccgtccagtctgtcctccacagggctccgggggcggggttcttcatgcccagcactgactctgcccctccctccccctcctgggcccccaggagacggtcccactccccagccgggacgtttcctcctgtggctttgtttttctgcattttctgcaccaaatggattttctgtggttttcctgaactcaccacactcttcaaacctctaccttctgcccatattgttccctccagttacaatgcccgtcccctcctctcagtcaagccatctcagtccctgtaagctaccccatcacagccttagtctggggccaaaaggcccactgtcaactcatagtgggccattcctgtctccacacgcttggatcagttatttcttctctctgagaatcaatttttgcattcagaaagtagaatacatggaaaaacccactgtatgtgcttttttgtgatgattaaatgcaatattgtgcataaagagcttagcatataatctctcacaagttagctaatttattgggtggatataatatgttacatagcctgggtgccatggctcacgcctgtaatcccagcactttgggaggccgaggtgggtggatcacgtgagatcagaagttagagaccagcctggccaatgtggcaaaaccccgtcttaccacaaaaaaatacttagctgggcgtgatggtgtgcacctgtgatcacagctactctggagggtgaggcaagagaatcacttgaacccaggaggcagaagttgcagtgagccgagatcacatcactgcactctggccttggcgacagagtgagactctgtctcaaaaaaaaaaaaatatatatatatatagatagatagatagatagatagatatagatatacacacacatactaataatatgttatataacatattatatacttaaatatatatacaaattatatataaagtatcaatatatgcttttttttttttgtgagggagttttgctcttgttgcccaggctggagtgcagtggcgtggtctcggctcactgcaacctccgcctcccgggttcaagcgattttcctgcctcagcctcccaagtagctgggattacaggcatgcgccaccacgcctggctaattttgtatttttagtagagatggggtttctccatgttggtcaggctggtctcgaactcccaacctcaggtgatctgcccacctcggcctcccaaagtgctgtgattacaggtttgagccaccgcacccggcctacttttttttttttttttttttttttgagatggaatggagtttcactcttgttggccaggctgcagtgcaatggcaaggtctcggcttactacaacctcatcctcctgggttcaagcaattctcctgcctcagcctcacgagtagctaggactacaggcgcccaccaccatgccaggcaaatttttgtatttttagtagagaaggagtttcaccatgttgaccaggctggtctcgaactcctgacctcaggcgatccacccacctcggcctcccaaagtgctgggattacaggtgtgagccactgtgccaggcttctaaaatttttttttttttttttttgagacatagcctccctctgtcgcccaggctggaaagcagtggtgcagtctcagctcacggcaacctccgcttcctgggttcaatcgactcttgtgcctcagactcccgagtagctgggattacaggcgtgccccaccatgcccggctaagtatttttttggtagagatggggtttcgccatgttggccaggctggtctcaaactcctgacctcaagtgatctgccagcttcagcctcccaaagtgctgctattacaggcgtgagccactgcatccagcctactgatatatacttattaactccacagaaatctttgtaccccagtaggatttactttgtctttgttttaacctgcttggcgtttcctccattaaagctcatctcactgagacctgtccttccccaggtgaagaatttcatgacgtggaggatgcagagacctacaaaaagatgctggctcgggacgagcggcgtttccgggtggccgaccaggatggggactcgatggccactcgagaggagctgacagccttcctgcaccccgaggagttccctcacatgcgggacatcgtgattgctgtgagtggcggctggggaaccctgtcccccacacccttccggggacccaggcttccggttcaggtcctgcttcccagcaccgacctgggggcaggcagtcacctggttctccagcatcttgccggggaagccaggccgcaaacacaggtcagaggacggtgacctttcccacctccactctcagtcccctggggtctgatctctcctcgtctggccttcccctccctgtgctcgcatttggggtccaggcccccagccttccctcagacagccactgtgccccaccttatacaatttcttttctttcttttttttttttgagacatagtctccctcggttgcccaggctggaaagcagtggtgcgatctcagctcactgcaacctccgcctcccgggttcaagcaattctcctgcctcagcgtcccgagtagctgggattacaggggcgcaccaccacacctggctaatttttagtagagacagggtttcttcatgttggttaggctggtctcaaacttttgacctcaggtgatccgcccgcctcagcctcccaaagtgctaggattataggcatgagccaccgtgcccggccaatttttaatttttttttctagtagagatgggatttcaccatgttggccaggctggtcttgaactccggacctcaggtgatctgcccaccttggcctcccaaagtgctgggattacaggcatgagccaccgtgcctggccccaaaatgtgctttgttctatagaagaaggctttttttttctttccttcctcttttttttttttagtttttgagacagggcctaactctgttacccagaccggagggcagtggctcaatctcagctcactgcaacctcaaactcttgggctcaagtaatcctcccacctccgccttctgagaagctgaggccaccaggcacaagccaccatgtctggctaatgtttttttttttttttttttcagacggagtctcgctctgtcgcccatgctggagtgcagtgatgcgatctcggctcactgcaagctctgcctcccgggttcactccattcttctgcctcagcctcccgagtggctgggactacaggcgcatgccgccatgcccggctaatttttttttttttttttttagtagagacggggtttcactgtgttagccaggatggtctcgatctcctgacctcgtgatccacccacctcggccttccaaagtgctgggattacagttgtgagccaccacgcccggccatgcctggctaatttttaaattttgtgtagagacagggtctcgccatgttgcccaggctagtctcaaaatcctgggctcaatcgatcctcccgcctcggcctcccaaagtgctgggattacaggtgtgagccaccgtgcccagcccacagtcttatagtgaatgagtttggatataaattggggaacaaaagtcgttttgctccagaagggcacaaaaacaaggagcggaggtgtccagtgtgtcagaagaagccccaaagtgcaccatgttcacacataaagcagaggtaggttctaggctctgctaaacataaaggagcttctccccttggagggtgtttgctggggcttgagcaagttattagggacccaggacagctccccttgtgagcaacaaacctgtgcactgctggacattagggcctctgggctctggccccgtaaatgcaggtggtgcctgtttgatgataacatcccagaaagcccgcaaatttcccacatgccacctgttgagactgctctactgcctgcctcccacagctgcccggagctaaatttccaagagaacctctcactcttaccccaggggtccagcctcccccaggagccagggtcatcggcccccagcctcaatgcccctttctcctccaggaaaccctggaggacctggacagaaacaaagatggctatgtccaggtggaggagtacatcggtgagtgggccccaatttcttcttgggatgcctgtcctctctcaggcatgggcagggttggtgggggtagccggaggtacatcacccatcatcagagaacagtggccctcagacatgggggcaggggcactcactgcttgagttcatttagccacccaacatttattgagcacctactctataccagacacaaaaagcatctattgagcacctgctgtgtgtcacgcccaggatacacttactgagcacctactgcatatgggtggttgcgcctacagcagacaataacacagacaaatcccttcttgggggtctggagtttctacaaaatcaaggctcaggggctatgtttagttgagaatgggggtcaggaagctggtcttgaacctgcgcctgcacagcaaacactgtttcctgttacatgggatgttgggggatcctcacaataccccctgagttctgagttctccctgggcccctgccactgtccctgcccctagcgggcaaaaaatttagtcaaggagaacgctaacaaggaattttttttttttttttttttttttgagacagtttttttctgttgctcaggctggagcgcggtggcgtggtatcggctctctgcaacctccgcctcccgggctcaagagattcttctgcctcagcctcctgagtagctgggattactggtgcctgccaccacgcctggctaatttttgtatttttagtggagatgggatttcaccatgttggccaggctggtctagaactcccaacctcaggtgatctgctcatcttggcctcccaaagtggtgggattacaggcgtgagcaaccacgcccggccaggaatttttttttaagtttgaaaacattgtttattcttttatttttttctgaccacatcctacatgagaaaacatttttctttctttccttctctcactttctttctttctgtcactctttctttctatctgtctgtctgtgtgtgtgtgtgtctctctctttattaaatagagatgggttctcactatattgtccaggctggtctagaactcctgggctcaagcaatcttcccaccttggcctcccaaagagctgagattacaggcatgaatcaccacacctggctgagagaacatttttatatttttttcttaagagatggggttgggccaggcgcagtggctcatacctgtaatcccagcactttgggaggctgagatggatggatcatgaagtcaggagttcgagaccagcctggccaagatggtgtactaaaaatacaaaaattagccgggcctgttggcaggagcctgtaatcccagttactctggggactgaggcaagagaatctctggaacccgggaggcaaaggttgcagtgagctgtaatcgcgccattgcacttcagtctgggcaacaagagcgaaactccatcttaaaaaaaaaaaaaaaagatggggtcttgccctgtcgcccaggctggagtgcagtggggtgatcttggctcactgcagccttgacctcctgggctcaagggatcctcccacctcagcctcccaagtagctgagactccaggcacagtgcggcctctggagatacttctggttgtcacttctagaggggtgaggatgctactgacgtctcctggatagagcccagagatgctccttagcattctccggtgcacaggacaggccctgacaacaaggaggtcacaggccccagatgccagcagctgtccccatctctttttttttttttttgagacagagtctcgctattgtcgcccaggctggagtgcagtggcatgatctcggctcactgcaacctccgcctcccaggttcaagcaattctcttgtttcagcctcctgagtaggtgggattatgggcgtgcactaccacgcctggctaattttgtatttttagtagagacggggtttcaccattttggtcaggctggtcttgaactcttgacctcaggttatccacctgcctcggcctcccaaagtgctgggattacaggcgcgaaccaccgcacccagcctatcttctcaattacttcatcccattaagacttcatcttccattcctcagctctaccaggcagacactgtcactatcctccattatatagatgacacctgaggcccaggggggttgaagggatctgctgaagacaatgggaagaggcagggctaggatttgaaccttggtgtttgggctctggcacctatttcttaactcctccccaaccagtaacatatcaaaatgtataaaattggggtgggtgccatggctnacgcctgtattcccagcactttaggaggccaaggtgggtggatcacttgaggtcaagagttcaagaccagcctggccaacatgatgaaaccctgtctctactaaaactagaaaaattagctgggcgtggtggcgggtacctgtaatcccagctacttgggaggctgaggcaggagaatcacttgaacccagaaggtgcaggttgcagtgagctgagattgtgtcactgcactccagcctgtaatcccagcactttgggagactgaggcaggtggatcatggggtcaggagtttgagagcagcctggccaagatggtgaaaccccgcctctactaaaaatataaaaattagctgggtgtggtggcacgcacctgtaatcccagctacttgggaggctgaggcaggagaatcgcttgaacctgggaggcagaggttgcagtgagctgagattgcgccattgcactacagcctgggcaacaagagcgaaactccatcttaaaaaaaaaaagggggtggggtcttgccctgttgcccaggctggagtgcagtggggtaatcacagcgagactccatctcaaaaaaaaaaaaaaaaatttgtagaaacagggtcttgctatgttgcccaggttggtcttgaatcttggcctcaaatgaccctcctgcctgggtctcccaaagtgttggattacagtcatcactgcacctggcatctctgtcacctgctaactggccagaaacatcctcgcacgttcctcctgcatagacattgcttactatgtgccaggcactgccttgcccattgcacacactacttcacgtgtgtgtcaatgaaatcaatcaatcatgcaatcaaccaatcaaatgaatttcacctgtaacacaaatgattgcttataagtcaggttttgttactaattccatcgaaacagaagaggacgctgaggcgagagagagggtagtgagttgctcaagggcctggttcaggaagtatcagagccaggatcagcccccagctccactagacccccagagctgcccctgaccttgtcccctctgtcccggcccccagcggatctgtactcagccgagcctggggaggaggagccggcgtgggtgcagacggagaggcagcagttccgggacttccgggatctgaacaaggatgggcacctggatgggagtgaggtgggccactgggtgctgccccctgcccaggaccagcccctggtggaagccaaccacctgctgcacgagagcgacacggacaaggtgcagtgacggggcctcggcaggagcgaggagcgggtgggcattgcgggccagactccagaaaggagcaagacctgggcctggagctcagggtcccttttaggtgggataaaaaaagagggacagagagagggaggaaaagagagggcacggagccccagaaagagagggggacagagacccagagacccaaagagagaaggacagggaccaagacggggacagattcagagagaaagggacagaggcccagagaacaagggtcccagagacttcgggacacgcttggatgcagggagggcttttgaaagcagggccgtgttgtcccctctgaaccctgaccctccctccaggatgggcggctgagcaaagcggaaatcctgggtaattggaacatgtttgtgggcagtcaggccaccaactatggcgaggacctgacccggcaccacgatgagctgtgagcaccgcgcacctgccacagcctcagaggcccgcacaatgaccggaggaggggccgctgtggtctggccccctccctgtccaggccccgcaggaggcagatgcagtcccaggcatcctcctgcccctgggctctcagggaccccctgggtcggcttctgtccctgtcacacccccaaccccagggaggggctgtcatagtcccagaggataagcaatacctatttctgactgagtctcccagcccagacccagggacccttggccccaagctcagctctaagaaccgccccaacccctccagctccaaatctgagcctccaccacatagactgaaactcccctggccccagccctctcctgcctggcctggcctgggacacctcctctctgccaggaggcaataaaagccagcgccggga

SEQ ID NO: 3 - protein, 124 aa, synthetic construct
  REGION 1..124: note = Synthetic sequence; Dupilumab HCVR
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 4 - protein, 112 aa, synthetic construct [sequence omitted]

SEQ ID NO: 5 - protein, 8 aa, synthetic construct
  REGION 1..8: note = Synthetic sequence; Dupilumab HCDR1
GFTFRDYA

SEQ ID NO: 6 - protein, 8 aa, synthetic construct
  REGION 1..8: note = Synthetic sequence; Dupilumab HCDR2
ISGSGGNT

SEQ ID NO: 7 - protein, 18 aa, synthetic construct
  REGION 1..18: note = Synthetic sequence; Dupilumab HCDR3
AKDRLSITIRPRYYGLDV

SEQ ID NO: 8 - protein, 11 aa, synthetic construct
  REGION 1..11: note = Synthetic sequence; Dupilumab LCDR1
QSLLYSIGYNY

SEQ ID NO: 9 - skipped

SEQ ID NO: 10 - protein, 9 aa, synthetic construct
  REGION 1..9: note = Synthetic sequence; Dupilumab LCDR3
MQALQTPYT

SEQ ID NO: 11 - protein, 451 aa, synthetic construct
  REGION 1..451: note = Synthetic sequence; Dupilumab heavy chain
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 219 aa, synthetic construct
  REGION 1..219: note = Synthetic sequence; Dupilumab light chain
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0151426 A1
(43) Pub. Date: May 18, 2023

(54) Reticulocalbin-3 (RCN3) Variants And Treatment Of Asthma With Interleukin-4 Receptor Alpha (IL4R) Antagonists

(71) Applicant: Regeneron Pharmaceuticals, Inc., Tarrytown, NY (US)

(72) Inventors: Charles Paulding, Tarrytown, NY (US); Shan Chen, Tarrytown, NY (US)

(21) Appl. No.: 17/935,785

(22) Filed: Sep. 27, 2022

Related U.S. Application Data
(60) Provisional application No. 63/250,546, filed on Sep. 30, 2021.

(51) Int. Cl. C12Q 1/6883; A61K 39/395; A61P 11/06

(52) U.S. Cl. CPC C12Q 1/6883; A61K 39/3955; A61P 11/06; C12Q 2600/158; C12Q 2600/106

(57) ABSTRACT

The present disclosure provides methods of identifying subjects having a risk of developing asthma exacerbation and methods of treating subjects having asthma and who are undergoing or will be undergoing treatment with an IL4R alpha antagonist and/or the IL13 blocking agent.

### REFERENCE TO SEQUENCE LISTING

This application includes a Sequence Listing submitted electronically as an XML file named 381203563SEQ, created on Sep. 23, 2022, with a size of 46 kilobytes. The Sequence Listing is incorporated herein by reference.

### FIELD

The present disclosure relates generally to methods of decreasing the asthma exacerbation rate in a subject having asthma and methods of treating subjects having asthma and who are undergoing or will be undergoing treatment with an IL4R alpha antagonist.

### BACKGROUND

Asthma is an inflammatory disease of the airways of the lungs. The condition is characterized by variable and recurring symptoms, reversible airflow obstruction, and bronchospasms that are easily triggered. The diagnosis of asthma can involve spirometry lung function testing, including determination of the forced expiratory volume in one second (FEV1), and the peak expiratory flow rate, as well as assessment of the annualized exacerbation rate. Treatments of asthma include administration of medications that are fast acting or effective in the longer term. Salbutamol is the mainstay of fast-acting medications, whereas inhaled corticosteroids were for many years the mainstay of long-term therapies. More recently, antibodies such as mepolizumab, dupilumab, and omalizumab, have been used in connection with specific types of asthma. DUPIXENT®, which comprises dupilumab, is currently approved for subjects that are 12 years-old and older.

Reticulocalbin-3 or EF-Hand Calcium-Binding Protein RLP49 (“RCN3”) is an endoplasmic reticulum lumen protein localized to the secretory pathway. Knockout of the RCN3 gene in mice is lethal for neonates and is associated with atelectasis-induced neonatal respiratory distress, failure of maturation of alveolar epithelial type II cells (AECIIs), and dramatic reductions in surfactant proteins A and D. Atelectasis is a complete or partial collapse of the entire lung or area (lobe) of the lung. Atelectasis occurs when the tiny air sacs (alveoli) within the lung become deflated or possibly filled with alveolar fluid. Atelectasis is one of the most common breathing (respiratory) complications after surgery. In vitro studies to reduce RCN3 expression results in blunting of the secretion of surfactant proteins (see, Jin et al., Am. J. Respir. Cell Mol. Biol., 2016, 54, 410-23). In addition, selective deletion of RCN3 in AECIIs in adult mice results in exacerbated pulmonary fibrosis and reduced lung mechanics after exposure to bleomycin (see, Jin et al., Am. J. Respir. Cell Mol. Biol., 2018, 59, 320-333).

### SUMMARY

The present disclosure provides methods of decreasing the asthma exacerbation rate in a subject having asthma, the methods comprising: determining whether the subject has a reticulocalbin-3 (RCN3) variant nucleic acid molecule by: obtaining or having obtained a biological sample from the subject; and performing or having performed a sequence analysis on the biological sample to determine if the subject has a genotype comprising the RCN3 variant nucleic acid molecule; and administering or having administered an IL4R alpha antagonist and/or an IL13 blocking agent to a subject that is heterozygous or homozygous for the RCN3 variant nucleic acid molecule, thereby decreasing the asthma exacerbation rate in the subject.

The present disclosure also provides methods of treating a subject having asthma and who is undergoing or will be undergoing treatment with an IL4R alpha antagonist and/or an IL13 blocking agent, the method comprising: determining whether the subject has a reticulocalbin-3 (RCN3) variant nucleic acid molecule by: obtaining or having obtained a biological sample from the subject; and performing or having performed a sequence analysis on the biological sample to determine if the subject has a genotype comprising the RCN3 variant nucleic acid molecule; and administering or continuing to administer the IL4R alpha antagonist and/or the IL13 blocking agent in a standard dosage amount to a subject that is RCN3 reference; and administering or continuing to administer to a subject that is heterozygous or homozygous for the RCN3 variant nucleic acid molecule the IL4R alpha antagonist and/or the IL13 blocking agent in an amount that is the same as or greater than a standard dosage amount and/or an RCN3 agonist; wherein the presence of a genotype in a subject that is homozygous for the RCN3 variant nucleic acid molecule indicates the subject has an increased risk of asthma exacerbation compared to a subject having a genotype that is heterozygous for the RCN3 variant nucleic acid molecule, and the presence of a genotype that is heterozygous for the RCN3 variant nucleic acid molecule indicates the subject has an increased risk of asthma exacerbation compared to a subject having a genotype that is RCN3 reference.

The present disclosure also provides methods of identifying a subject having a risk of developing asthma exacerbation, the method comprising: determining or having determined the presence or absence of a reticulocalbin-3 (RCN3) variant nucleic acid molecule in a biological sample obtained from the subject; wherein when the subject is heterozygous or homozygous for the RCN3 variant nucleic acid molecule, the subject has an increased risk of developing asthma exacerbation compared to a subject that is RCN3 reference.

The present disclosure also provides methods of treating a subject having asthma or at risk of developing asthma and who is undergoing or will be undergoing treatment with an IL4R alpha antagonist and/or an IL13 blocking agent, the methods comprising: determining or having determined the subject's RCN3 gene expression score (RGES), wherein the RGES comprises a value determined from gene expression in a sample from the subject, and when the subject's RGES is greater than a threshold RGES determined from a reference population of subjects without asthma, administering or continuing to administer to the subject the IL4R alpha antagonist and/or the IL13 blocking agent in an amount that is the same as or greater than a standard dosage amount and/or an RCN3 agonist.

The present disclosure also provides an amount of an IL4R alpha antagonist and/or the IL13 blocking agent that is the same as or greater than a standard dosage amount and/or an RCN3 agonist, for use in the treatment of asthma in a subject having a risk of developing asthma exacerbation, wherein the subject is identified as having an RCN3 variant genomic nucleic acid molecule, or the complement thereof, wherein the RCN3 variant genomic nucleic acid molecule has a nucleotide sequence comprising a guanine at a position corresponding to position 13,482 according to SEQ ID NO:2, or the complement thereof.

### BRIEF DESCRIPTION OF THE DRAWINGS

The accompanying figures, which are incorporated in and constitute a part of this specification, illustrate several features of the present disclosure.

The patent or application file contains at least one drawing executed in color. Copies of this patent or patent application publication with color drawing(s) will be provided by the Office upon request and payment of the necessary fee.

FIG. 1 shows association of 19:49541484:C:G (rs113886122) with reduced RCN3 expression in Genotype-Tissue Expression (GTEX) when considering expression quantitative trait loci (eQTLs) and splicing quantitative trait loci (sQTLs).

FIG. 2 shows significant association of 19:49541484:C:G (rs113886122) with exacerbation only in dupilumab-treated subjects.

FIG. 3 shows association of 19:49541484:C:G (rs113886122) with an increased annualized exacerbation rate in dupilumab-treated asthma subjects.

FIG. 4 shows association of 19:49541484:C:G (rs113886122) with increased exacerbation in dupilumab-treated subjects as well as decreased baseline lung function.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is not intended that any method or aspect set forth herein be construed as requiring that its steps be performed in a specific order. Accordingly, where a method claim does not specifically state in the claims or descriptions that the steps are to be limited to a specific order, it is not intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or other tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

A common variant in the RCN3 gene associated with a risk of developing asthma exacerbation in humans has been identified in accordance with the present disclosure. For example, a genetic alteration that changes the cytosine at position 13,482 in the RCN3 reference genomic nucleic acid molecule (see, SEQ ID NO:1) to a guanine has been observed to indicate that the subject having such an alteration may have a risk of developing asthma exacerbation. Prior to this disclosure, it was believed that no variants of the RCN3 gene or RCN3 protein had any association with an increased annualized exacerbation rate for asthma. Altogether, the genetic analyses described herein surprisingly indicate that the RCN3 gene and, in particular, a variant in the RCN3 gene, associates with a risk of developing asthma exacerbation. Therefore, subjects that have an RCN3 variant nucleic acid molecule that have a risk of developing an asthma exacerbation may be treated such that the asthma exacerbation rate is reduced, the symptoms thereof are reduced, and/or development of symptoms is repressed. Accordingly, the present disclosure provides methods of leveraging the identification of such variants in subjects to identify or stratify risk in such subjects of developing an increased annualized exacerbation rate for asthma or to diagnose subjects as having a risk of developing asthma exacerbation such that subjects at risk or subjects with active disease may be treated accordingly.

For purposes of the present disclosure, any particular subject can be categorized as having one of three RCN3 genotypes: i) RCN3 reference; ii) heterozygous for an RCN3 variant nucleic acid molecule; or iii) homozygous for an RCN3 variant nucleic acid molecule. A subject is RCN3 reference when the subject does not have a copy of an RCN3 variant nucleic acid molecule. By way of a non-limiting example, the nucleotide sequence set forth in SEQ ID NO:1 is RCN3 reference because the nucleotide at position 13,482 comprises a cytosine rather than a guanine. A subject is heterozygous for an RCN3 variant nucleic acid molecule when the subject has a single copy of an RCN3 variant nucleic acid molecule. Without being limited to any particular theory, it is believed that the presence of an RCN3 variant nucleic acid molecule ultimately results in the increased annualized exacerbation rate observed. Therefore, as used herein, an RCN3 variant nucleic acid molecule is any RCN3 nucleic acid molecule (such as, a genomic nucleic acid molecule, an mRNA molecule, a noncoding RNA, or a cDNA molecule) that results in altered (or decreased) expression of the RCN3 mRNA, a noncoding RNA, and/or protein with respect to that observed in RCN3 reference subjects. By way of a non-limiting example, the nucleotide sequence set forth in SEQ ID NO:2 is an RCN3 variant nucleic acid because the nucleotide at position 13,482 comprises a guanine rather than a cytosine (i.e., rs113886122). A subject is homozygous for an RCN3 variant nucleic acid molecule when the subject has two copies of an RCN3 variant nucleic acid molecule.

For subjects that are genotyped or determined to be heterozygous or homozygous for an RCN3 variant nucleic acid molecule, such subjects have a risk of developing asthma exacerbation. For subjects that are genotyped or determined to be heterozygous or homozygous for an RCN3 variant nucleic acid molecule, such subjects can be treated with an amount of an IL4R alpha antagonist and/or an IL13 blocking agent that is the same as or greater than a standard dosage amount. In addition, for subjects that are genotyped or determined to be heterozygous or homozygous for an RCN3 variant nucleic acid molecule, such subjects can be treated with an amount of an IL4R alpha antagonist and/or an IL13 blocking agent that is the same as or greater than a standard dosage amount and/or with an RCN3 agonist. In some embodiments, the subjects can also be treated with a therapeutic agent that treats or inhibits asthma exacerbation.

In any of the embodiments described throughout the present disclosure, the RCN3 variant nucleic acid molecule can be any RCN3 variant nucleic acid molecule described herein.

In any of the embodiments described herein, exacerbations can be considered as a worsening of asthma requiring an emergency department (ED)/hospital admission or oral corticosteroid (OCS) treatment. In some embodiments, a “severe exacerbation event” is defined as a deterioration of asthma requiring use of systemic corticosteroids for 3 days and/or hospitalization or emergency room visit because of asthma requiring systemic corticosteroids.

The present disclosure provides methods of decreasing the asthma exacerbation rate in a subject having asthma. In some embodiments, the methods comprise determining whether the subject has a reticulocalbin-3 (RCN3) variant nucleic acid molecule. This determination can be carried out by obtaining or having obtained a biological sample from the subject and performing or having performed a sequence analysis on the biological sample to determine if the subject has a genotype comprising the RCN3 variant nucleic acid molecule. When the subject is heterozygous or homozygous for the RCN3 variant nucleic acid molecule, the subject is administered an IL4R alpha antagonist and/or an IL13 blocking agent, thereby decreasing the asthma exacerbation rate in the subject.

The present disclosure also provides methods of treating a subject having asthma and who is undergoing or will be undergoing treatment with an IL4R alpha antagonist and/or an IL13 blocking agent. In some embodiments, the subject is undergoing treatment with an IL4R alpha antagonist and/or an IL13 blocking agent. In some embodiments, the subject has not yet but will be undergoing treatment with an IL4R alpha antagonist and/or an IL13 blocking agent. In some embodiments, the methods comprise determining whether the subject has an RCN3 variant nucleic acid molecule. This determination can be carried out by obtaining or having obtained a biological sample from the subject and performing or having performed a sequence analysis on the biological sample to determine if the subject has a genotype comprising the RCN3 variant nucleic acid molecule. When the subject is RCN3 reference, the IL4R alpha antagonist and/or an IL13 blocking agent is administered or continued to be administered in a standard dosage amount. When the subject is heterozygous or homozygous for the RCN3 variant nucleic acid molecule, the IL4R alpha antagonist and/or an IL13 blocking agent is administered or continued to be administered in an amount that is the same as or greater than a standard dosage amount and/or an RCN3 agonist is administered or continued to be administered. The presence of a genotype having the RCN3 variant nucleic acid molecule indicates the subject has an increased risk of developing asthma exacerbation. The presence of a genotype in a subject that is homozygous for the RCN3 variant nucleic acid molecule indicates the subject has an increased risk of asthma exacerbation compared to a subject having a genotype that is heterozygous for the RCN3 variant nucleic acid molecule. The presence of a genotype that is heterozygous for the RCN3 variant nucleic acid molecule indicates the subject has an increased risk of asthma exacerbation compared to a subject having a genotype that is RCN3 reference.

The present disclosure also provides methods of treating a subject having asthma with an IL4R alpha antagonist and/or an IL13 blocking agent, wherein the subject is heterozygous or homozygous for an RCN3 variant nucleic acid molecule. The subject can be administered the IL4R alpha antagonist and/or an IL13 blocking agent in an amount that is the same as or greater than a standard dosage amount and can also be administered an RCN3 agonist.

In any of the embodiments described herein, the treatment methods can also further comprise administering or continuing to administer to the subject a therapeutic agent that treats or inhibits asthma exacerbation.

In any of the embodiments described herein, the RCN3 variant nucleic acid molecule is a genomic nucleic acid molecule having a nucleotide sequence comprising a guanine (or a thymine) at a position corresponding to position 13,482 according to SEQ ID NO:2. In some embodiments, the RCN3 variant nucleic acid molecule is a genomic nucleic acid molecule having a nucleotide sequence comprising a guanine at a position corresponding to position 13,482 according to SEQ ID NO:2. The variant nucleic acid molecules disclosed herein can also include any genetic variants, regardless of their genomic annotation, in proximity to (for example, up to 10 Mb around the gene) or in linkage disequilibrium with the RCN3 gene that show a non-zero association with asthma in a genetic association analysis.

In any of the embodiments described herein, the asthma can be childhood asthma, allergic asthma, non-allergic asthma, exercise-induced asthma, occupational asthma, adult-onset asthma, or nocturnal asthma. In some embodiments, the asthma is childhood asthma. In some embodiments, the asthma is allergic asthma. In some embodiments, the asthma is non-allergic asthma. In some embodiments, the asthma is exercise-induced asthma. In some embodiments, the asthma is occupational asthma. In some embodiments, the asthma is adult-onset asthma. In some embodiments, the asthma is nocturnal asthma.

In some embodiments, the subject is an adult. In some embodiments, the subject is an infant under the age of 2. In some embodiments, the subject is an infant born prematurely. Infants that are heterozygous or homozygous for an RCN3 variant nucleic acid molecule can be further treated with surfactant. In some embodiments, the subject is from about 6 years-old to about 12 years-old. In some embodiments, the subject is at or over the age of 12.

In some embodiments, the subject is RCN3 reference. In some embodiments, the subject is heterozygous for an RCN3 variant nucleic acid molecule. In some embodiments, the subject is homozygous for an RCN3 variant nucleic acid molecule.

In some embodiments, the subject is RCN3 reference, and the subject is administered or continued to be administered the IL4R alpha antagonist and/or the IL13 blocking agent in a standard dosage amount. In some embodiments, the subject is heterozygous or homozygous for the RCN3 variant nucleic acid molecule, and the subject is administered or continued to be administered the IL4R alpha antagonist and/or the IL13 blocking agent in an amount that is the same as or greater than a standard dosage amount. In some embodiments, the subject is heterozygous or homozygous for the RCN3 variant nucleic acid molecule, and the subject is administered or continued to be administered the IL4R alpha antagonist and/or the IL13 blocking agent in an amount that is the same as or greater than a standard dosage amount and/or an RCN3 agonist.

Detecting the presence or absence of an RCN3 variant nucleic acid molecule in a biological sample obtained from a subject and/or determining whether a subject has an RCN3 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the RCN3 variant nucleic acid molecule can be present within a cell obtained from the subject.

IL4R alpha antagonists include, but are not limited to, dupilumab and pitrakinra. IL13 blocking agents include, but are not limited to, dupilumab, tralokinumab, pitrakinra, and lebrikizumab. A standard dosage amount of dupilumab for adults and adolescents (12 years of age and older) is: i) an initial dose of 400 mg (two 200 mg injections) followed by 200 mg administered every other week; or ii) an initial dose of 600 mg (two 300 mg injections) followed by 300 mg administered every other week; or iii) for patients requiring concomitant oral corticosteroids or with co-morbid moderate-to-severe atopic dermatitis for which dupilumab is indicated, starting with an initial dose of 600 mg followed by 300 mg administered every other week. In some embodiments, the IL4R alpha antagonist is not an IL13 blocking agent. In some embodiments, the IL13 blocking agent is not an IL4R alpha antagonist. In some embodiments, the IL4R alpha antagonist and/or the IL13 blocking agent are separate antagonists. Where the IL4R alpha antagonist and/or the IL13 blocking agent are separate antagonists, a first separate antagonist is an IL4R alpha antagonist but not an IL13 blocking agent and a second separate antagonist is an IL13 blocking agent but not an IL4R alpha antagonist, i.e., two separate antagonists are administered.

In some embodiments, the IL4R alpha antagonist specifically binds to human IL-4Rα and comprises a heavy chain variable region (HCVR) comprising SEQ ID NO:3 and a light chain variable region (LCVR) comprising SEQ ID NO:4, a heavy chain complementarity determining region 1 (HCDR1) comprising SEQ ID NO:5, a HCDR2 comprising SEQ ID NO:6, a HCDR3 comprising SEQ ID NO:7, a light chain complementarity determining region 1 (LCDR1) comprising SEQ ID NO:8, a LCDR2 comprising SEQ ID NO:9, and a LCDR3 comprising SEQ ID NO:10. The full-length heavy chain of dupilumab is shown as SEQ ID NO: 311 and the full length light chain is shown as SEQ ID NO:12. Human anti-IL-4R antibodies can be generated as described in U.S. Pat. No. 7,608,693.

An exemplary RCN3 agonist is an RCN3 protein.

Examples of therapeutic agents that treat or inhibit acute asthma exacerbation include, but are not limited to, inhaled corticosteroids (ICS), alone or combined with long-acting β2-agonists (LABA), oral corticosteroids, dupilumab, mepolizumab, benralizumab, reslizumab, omalizumab, tezepelumab, and azithromycin. In some embodiments, an increased dose of ICS can be administered.

In some embodiments, the dose of the IL4R alpha antagonist and/or the IL13 blocking agent can be increased by about 10%, by about 20%, by about 30%, by about 40%, by about 50%, by about 60%, by about 70%, by about 80%, or by about 90% for subjects that are heterozygous or homozygous for an RCN3 variant nucleic acid molecule (i.e., greater than the standard dosage amount) compared to subjects that are RCN3 reference (who may receive a standard dosage amount). In some embodiments, the dose of the IL4R alpha antagonist and/or the IL13 blocking agent can be increased by about 10%, by about 20%, by about 30%, by about 40%, or by about 50%. In addition, the dose of the IL4R alpha antagonist and/or the IL13 blocking agent in subjects that are heterozygous or homozygous for an RCN3 variant nucleic acid molecule can be administered more frequently compared to subjects that are RCN3 reference.

Administration of the IL4R alpha antagonists, the IL13 blocking agents, the RCN3 agonists, and/or therapeutic agents that treat or inhibit asthma exacerbation can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the IL4R alpha antagonists, the IL13 blocking agents, the RCN3 agonists, and/or therapeutic agents that treat or inhibit asthma exacerbation can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in the increased annualized exacerbation rate, a decrease/reduction in the severity of the increased annualized exacerbation rate (such as, for example, a reduction or inhibition of development of an increased annualized exacerbation rate), a decrease/reduction in symptoms and increased annualized exacerbation rate-related effects, delaying the onset of symptoms and increased annualized exacerbation rate-related effects, reducing the severity of symptoms of increased annualized exacerbation rate-related effects, reducing the severity of an acute episode, reducing the number of symptoms and increased annualized exacerbation rate-related effects, reducing the latency of symptoms and increased annualized exacerbation rate-related effects, an amelioration of symptoms and increased annualized exacerbation rate-related effects, reducing secondary symptoms, preventing relapse to increased annualized exacerbation rate, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of an increased annualized exacerbation rate development/progression (such as, for example, a complete or partial avoidance/inhibition or a delay), and an increased survival time of the affected host animal, following administration of a therapeutic protocol. Treatment of an increased annualized exacerbation rate encompasses the treatment of subjects already diagnosed as having any form of an exacerbation at any clinical stage or manifestation, the delay of the onset or evolution or aggravation or deterioration of the symptoms or signs of an exacerbation, and/or preventing and/or reducing the severity of an exacerbation.

The present disclosure also provides methods of treating a subject having asthma or at risk of developing asthma and who is undergoing or will be undergoing treatment with an IL4R alpha antagonist and/or an IL13 blocking agent, the methods comprising: determining or having determined the subject's RCN3 gene expression score (RGES), wherein the RGES comprises a value determined from gene expression in a sample from the subject, and when the subject's RGES is greater than a threshold RGES determined from a reference population of subjects without asthma, administering or continuing to administer to the subject the IL4R alpha antagonist and/or the IL13 blocking agent in an amount that is the same as or greater than a standard dosage amount and/or an RCN3 agonist.

The present disclosure also provides methods of identifying a subject having a risk of developing asthma exacerbation. In some embodiments, the methods comprise determining or having determined the presence or absence of an RCN3 variant nucleic acid molecule in a biological sample obtained from the subject. When the subject lacks an RCN3 variant nucleic acid molecule (i.e., the subject is genotypically categorized as RCN3 reference), the subject does not have an increased risk of developing asthma exacerbation. When the subject has an RCN3 variant nucleic acid molecule (i.e., the subject is heterozygous or homozygous for a RCN3 variant nucleic acid molecule), the subject has an increased risk of developing asthma exacerbation.

It is believed that a single copy of an RCN3 variant nucleic acid molecule (i.e., heterozygous for an RCN3 variant nucleic acid molecule) renders the subject to have a greater risk of developing asthma exacerbation compared to a subject that is RCN3 reference. Having two copies of an RCN3 variant nucleic acid molecule (i.e., homozygous for an RCN3 variant nucleic acid molecule) may render the subject to have a greater risk of developing asthma exacerbation than having a single copy of an RCN3 variant nucleic acid molecule.

In some embodiments, the RCN3 variant nucleic acid molecule is a genomic nucleic acid molecule having a nucleotide sequence comprising a guanine at a position corresponding to position 13,482 according to SEQ ID NO:2. In some embodiments, the RCN3 variant nucleic acid molecule is a genomic nucleic acid molecule having a nucleotide sequence comprising a guanine at a position corresponding to position 13,482 according to SEQ ID NO:2.

Detecting the presence or absence of an RCN3 variant nucleic acid molecule in a biological sample obtained from a subject and/or determining whether a subject has an RCN3 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the RCN3 variant nucleic acid molecule can be present within a cell obtained from the subject.

In some embodiments, when the subject is RCN3 reference, the methods can further comprise administering or continuing to administer to the subject an IL4R alpha antagonist and/or the IL13 blocking agent in a standard dosage amount. In some embodiments, when the subject is heterozygous or homozygous for the RCN3 variant nucleic acid molecule, the methods can further comprise administering to the subject or continuing to administer to the subject an IL4R alpha antagonist and/or an IL13 blocking agent in a standard dosage amount or in an amount greater than a standard dosage amount and/or an RCN3 agonist. In some embodiments, the methods can also further comprise administering or continuing to administer to the subject a therapeutic agent that treats or inhibits asthma exacerbation.

In some embodiments, the IL4R alpha antagonist and/or the IL13 blocking agent is dupilumab. In some embodiments, the RCN3 agonist is an RCN3 protein.

In some embodiments, the subject is RCN3 reference. In some embodiments, the subject is heterozygous for an RCN3 variant nucleic acid molecule. In some embodiments, the subject is homozygous for an RCN3 missense variant nucleic acid molecule.

In some embodiments, the subject examined for risk is an adult. In some embodiments, the subject examined for risk is an infant under the age of 2. In some embodiments, the subject examined for risk is an infant born prematurely.

The present disclosure also provides methods of detecting the presence or absence of an RCN3 variant nucleic acid molecule in a biological sample obtained from a subject. It is understood that gene sequences within a population and RNA molecules (whether mRNA or noncoding RNA) encoded by such genes can vary due to polymorphisms such as single nucleotide polymorphisms (SNPs). The sequences provided herein for the RCN3 variant nucleic acid molecules are only exemplary sequences. Other sequences for the RCN3 variant nucleic acid molecules are also possible.

The biological sample can be derived from any cell, tissue, or biological fluid from the subject. The biological sample may comprise any clinically relevant tissue such as, for example, a bone marrow sample, a tumor biopsy, a fine needle aspirate, or a sample of bodily fluid, such as blood, gingival crevicular fluid, plasma, serum, lymph, ascitic fluid, cystic fluid, or urine. In some embodiments, the biological sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any RCN3 variant nucleic acid molecule, preliminary processing designed to isolate or enrich the biological sample for the RCN3 variant nucleic acid molecule can be employed. A variety of techniques may be used for this purpose.

The present disclosure also provides methods of detecting an RCN3 variant nucleic acid molecule, or the complement thereof, in a subject. The methods comprise assaying a biological sample obtained from the subject to determine whether a nucleic acid molecule in the biological sample is an RCN3 variant nucleic acid molecule. In some embodiments, the RCN3 variant nucleic acid molecule, or the complement thereof, is a genomic nucleic acid molecule having a nucleotide sequence comprising a guanine at a position corresponding to position 13,482 according to SEQ ID NO:2, or the complement thereof. In some embodiments, the RCN3 variant genomic nucleic acid molecule has a nucleotide sequence comprising a guanine at a position corresponding to position 13,482 according to SEQ ID NO:2, or the complement thereof.

In some embodiments, the biological sample comprises a cell or cell lysate. Such methods can further comprise, for example, obtaining a biological sample from the subject comprising an RCN3 genomic nucleic acid molecule. Such assays can comprise, for example determining the identity of these positions of the particular RCN3 nucleic acid molecule. In some embodiments, the method is an in vitro method.

In some embodiments, the assay comprises sequencing at least a portion of the nucleotide sequence of the RCN3 nucleic acid molecule, or the complement thereof, in the biological sample. In some embodiments, the assay comprises sequencing at least a portion of the nucleotide sequence of the RCN3 genomic nucleic acid molecule in the biological sample, wherein the sequenced portion comprises a position corresponding to position 13,482 according to SEQ ID NO:2, or the complement thereof.

In some embodiments, the assay comprises sequencing at least a portion of the nucleotide sequence of the RCN3 genomic nucleic acid molecule, or the complement thereof, in the biological sample, wherein the sequenced portion comprises a position corresponding to position 13,482 according to SEQ ID NO:2, or the complement thereof. When the sequenced portion of the RCN3 genomic nucleic acid molecule in the biological sample comprises a guanine at a position corresponding to position 13,482 according to SEQ ID NO:2, or the complement thereof, then the RCN3 genomic nucleic acid molecule in the biological sample is an RCN3 variant genomic nucleic acid molecule.

In some embodiments, the assay comprises: a) contacting the biological sample with a primer hybridizing to a portion of the nucleotide sequence of the RCN3 genomic nucleic acid molecule, or the complement thereof, that is proximate to a position corresponding to position 13,482 according to SEQ ID NO:2, or the complement thereof; b) extending the primer at least through the position of the nucleotide sequence of the RCN3 genomic nucleic acid molecule, or the complement thereof, corresponding to position 13,482 according to SEQ ID NO:2, or the complement thereof; and c) determining whether the extension product of the primer comprises a guanine at a position corresponding to position 13,482 according to SEQ ID NO:2, or the complement thereof.

In some embodiments, the assay comprises: a) contacting the biological sample with a primer hybridizing to a portion of the nucleotide sequence of the RCN3 genomic nucleic acid molecule, or the complement thereof, that is proximate to a position corresponding to position 13,482 according to SEQ ID NO:2, or the complement thereof; b) extending the primer at least through the position of the nucleotide sequence of the RCN3 genomic nucleic acid molecule, or the complement thereof, corresponding to position 13,482 according to SEQ ID NO:2, or the complement thereof; and c) determining whether the extension product of the primer comprises a guanine at a position corresponding to position 13,482 according to SEQ ID NO:2, or the complement thereof;.

In some embodiments, the assay comprises sequencing the entire nucleic acid molecule. In some embodiments, only RCN3 genomic nucleic acid molecules are analyzed.

In some embodiments, the assay comprises: a) amplifying at least a portion of the RCN3 nucleic acid molecule, or the complement thereof, in the biological sample, wherein the amplified portion comprises a guanine at a position corresponding to position 13,482 according to SEQ ID NO:2, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising a guanine at a position corresponding to position 13,482 according to SEQ ID NO:2, or the complement thereof; and d) detecting the detectable label.

In some embodiments, the assay comprises: a) amplifying at least a portion of the RCN3 genomic nucleic acid molecule, or the complement thereof, in the biological sample, wherein the portion comprises a guanine at a position corresponding to position 13,482 according to SEQ ID NO:2, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising a guanine at a position corresponding to position 13,482 according to SEQ ID NO:2, or the complement thereof; and d) detecting the detectable label.

In some embodiments, the assay comprises: contacting the RCN3 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the RCN3 nucleic acid molecule, or the complement thereof, comprising a guanine at a position corresponding to position 13,482 according to SEQ ID NO:2, or the complement thereof; and detecting the detectable label.

In some embodiments, the assay comprises: contacting the RCN3 genomic nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the RCN3 genomic nucleic acid molecule, or the complement thereof, comprising a guanine at a position corresponding to position 13,482 according to SEQ ID NO:2, or the complement thereof; and detecting the detectable label.

In some embodiments, the RCN3 nucleic acid molecule is present within a cell obtained from the subject.

Alteration-specific polymerase chain reaction techniques can be used to detect mutations such as SNPs in a nucleotide sequence. Alteration-specific primers can be used because the DNA polymerase will not extend when a mismatch with the template is present.

In some embodiments, the methods utilize probes and primers of sufficient nucleotide length to bind to the target nucleotide sequence and specifically detect and/or identify a polynucleotide comprising an RCN3 genomic nucleic acid molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether an RCN3 nucleic acid molecule (such as a genomic nucleic acid molecule), or complement thereof, within a biological sample comprises a nucleotide sequence comprising a guanine at a position corresponding to position 13,482 according to SEQ ID NO:2, the biological sample can be subjected to an amplification method using a primer pair that includes a first primer derived from the 5′ flanking sequence adjacent to a guanine at a position corresponding to position 13,482 according to 13,482 and a second primer derived from the 3′ flanking sequence adjacent to a guanine at a position corresponding to position 13,482 according to SEQ ID NO:2 to produce an amplicon that is indicative of the presence of the SNP at positions encoding a guanine at a position corresponding to position 13,482 according to SEQ ID NO:2. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a guanine at a position corresponding to position 13,482 according to SEQ ID NO:2, and at least 1, 2, 3, 4, 5, 6, 7, 8, 9, 10, or more nucleotides on each side of positions comprising a guanine at a position corresponding to position 13,482 according to SEQ ID NO:2.

PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6×sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides isolated nucleic acid molecules that hybridize to RCN3 variant genomic nucleic acid molecules. In some embodiments, such isolated nucleic acid molecules hybridize to RCN3 variant nucleic acid molecules under stringent conditions. Such nucleic acid molecules can be used, for example, as probes, primers, alteration-specific probes, or alteration-specific primers as described or exemplified herein.

In some embodiments, the isolated nucleic acid molecules hybridize to a portion of the RCN3 genomic nucleic acid molecule that includes a position corresponding to position 13,482 according to SEQ ID NO:2.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, the isolated alteration-specific probe or alteration-specific primer comprises at least about 15 nucleotides, wherein the alteration-specific probe or alteration-specific primer comprises a nucleotide sequence which is complementary to the nucleotide sequence of a portion of an RCN3 genomic nucleic acid molecule, or the complement thereof. In some embodiments, the portion comprises a position corresponding to position 13,482 according to SEQ ID NO:2.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to an RCN3 variant genomic nucleic acid molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of an RCN3 variant genomic nucleic acid molecule, wherein the portion comprises a position corresponding to position 13,482 according to SEQ ID NO:2, or the complement thereof.

In some embodiments, the isolated alteration-specific probe or alteration-specific primer comprises at least about 15 nucleotides, wherein the alteration-specific probe or alteration-specific primer comprises a nucleotide sequence which is complementary to the nucleotide sequence of a portion of an RCN3 nucleic acid molecule, or the complement thereof. In some embodiments, the portion comprises a position corresponding to position 13,482 according to SEQ ID NO:2, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within an RCN3 variant genomic nucleic acid molecule. The primers described herein can be used to amplify an RCN3 variant genomic nucleic acid molecule, or a fragment thereof.

The present disclosure also provides pairs of primers comprising any of the primers described above. For example, if one of the primers' 3′-ends hybridizes to a cytosine at a position corresponding to position 13,482 according to SEQ ID NO:1 (rather than a guanine) in a particular RCN3 nucleic acid molecule, then the presence of the amplified fragment would indicate the presence of an RCN3 reference genomic nucleic acid molecule. Conversely, if one of the primers' 3′-ends hybridizes to a guanine at a position corresponding to position 13,482 according to SEQ ID NO:2 (rather than a cytosine) in a particular RCN3 nucleic acid molecule, then the presence of the amplified fragment would indicate the presence of the RCN3 variant genomic nucleic acid molecule. In some embodiments, the nucleotide of the primer complementary to the guanine at a position corresponding to position 13,482 according to SEQ ID NO:2 can be at the 3′ end of the primer.

In the context of the present disclosure “specifically hybridizes” means that the probe or primer (such as, for example, the alteration-specific probe or alteration-specific primer) does not hybridize to a nucleotide sequence encoding an RCN3 reference genomic nucleic acid molecule.

In any of the embodiments described throughout the present disclosure, the probes (such as, for example, an alteration-specific probe) can comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well. In some embodiments, the support is a microarray.

The nucleotide sequence of an RCN3 reference genomic nucleic acid molecule is set forth in SEQ ID NO:1. Referring to SEQ ID NO:1, position 13,482 is a cytosine.

An RCN3 variant genomic nucleic acid molecule exists, wherein the cytosine at position 13,482 is replaced with a guanine. The nucleotide sequence of this RCN3 variant genomic nucleic acid molecule is set forth in SEQ ID NO:2.

The genomic nucleic acid molecules can be from any organism. For example, the genomic nucleic acid molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3× FLAG, 6× His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The isolated nucleic acid molecules, or the complement thereof, can also be present within a host cell. In some embodiments, the host cell can comprise the vector that comprises any of the nucleic acid molecules described herein, or the complement thereof. In some embodiments, the nucleic acid molecule is operably linked to a promoter active in the host cell. In some embodiments, the promoter is an exogenous promoter. In some embodiments, the promoter is an inducible promoter. In some embodiments, the host cell is a bacterial cell, a yeast cell, an insect cell, or a mammalian cell. In some embodiments, the host cell is a bacterial cell. In some embodiments, the host cell is a yeast cell. In some embodiments, the host cell is an insect cell. In some embodiments, the host cell is a mammalian cell.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, noncoding RNA molecules, and/or cDNA molecules disclosed herein, or vectors comprising the same. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, an RCN3 nucleic acid molecule comprising a nucleotide sequence comprising a guanine at a position corresponding to position 13,482 according to SEQ ID NO:2 means that if the nucleotide sequence of the RCN3 genomic nucleic acid molecule is aligned to the sequence of SEQ ID NO:2, the RCN3 sequence has a guanine residue at the position that corresponds to position 13,482 of SEQ ID NO:2. These phrases refer to an RCN3 nucleic acid molecule, wherein the genomic nucleic acid molecule has a nucleotide sequence that comprises a guanine residue that is homologous to the guanine residue at position 13,482 of SEQ ID NO:2.

As described herein, a position within an RCN3 genomic nucleic acid molecule that corresponds to position 13,482 according to SEQ ID NO:2, for example, can be identified by performing a sequence alignment between the nucleotide sequence of a particular RCN3 nucleic acid molecule and the nucleotide sequence of SEQ ID NO:2. A variety of computational algorithms exist that can be used for performing a sequence alignment to identify a nucleotide position that corresponds to, for example, position 13,482 in SEQ ID NO:2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides amounts of an IL4R alpha antagonist and/or the IL13 blocking agent that is greater than a standard dosage amount and/or an RCN3 agonist for use in the treatment of asthma in a subject having a risk of developing asthma exacerbation. wherein the subject is identified as having an RCN3 variant genomic nucleic acid molecule, or the complement thereof, wherein the RCN3 variant genomic nucleic acid molecule has a nucleotide sequence comprising a guanine at a position corresponding to position 13,482 according to SEQ ID NO:2, or the complement thereof.

The present disclosure also provides amounts of an IL4R alpha antagonist and/or the IL13 blocking agent that is greater than a standard dosage amount and/or an RCN3 agonist, or both, for use in the preparation of a medicament for treating asthma in a subject having a risk of developing an asthma exacerbation, wherein the subject is identified as having an RCN3 variant genomic nucleic acid molecule, or the complement thereof, wherein the RCN3 variant genomic nucleic acid molecule has a nucleotide sequence comprising a guanine at a position corresponding to position 13,482 according to SEQ ID NO:2, or the complement thereof.

In some embodiments, the RCN3 variant genomic nucleic acid molecule has a nucleotide sequence comprising a guanine at a position corresponding to position 13,482 according to SEQ ID NO:2, or the complement thereof.

In some embodiments, the IL4R alpha antagonist and/or the IL13 blocking agent is any of the IL4R alpha antagonists and IL13 blocking agents described herein, such as dupilumab. In some embodiments, the RCN3 agonist is any of the RCN3 agonists described herein, such as RCN3 protein.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1

### Genome-Wide Association Study (GWAS) Conducted for Annualized Exacerbation Rate for Asthmatic Subjects Who Received Dupilumab Treatment

To identify genes and pathways associated with the pathogenesis of asthma, a GWAS was conducted for multiple drug response endpoints. In particular, the annualized exacerbation rate for European asthmatic subjects who received dupilumab treatment in DRI12544 and EFC13579 trials was studied. The DRI and EFC European data were combined in the dupilumab arm, thereby providing a sample size of 654 subjects. A Manhattan plot for drug response end point, namely the annualized exacerbation rate, for subjects receiving dupilumab treatment was prepared. One locus near RCN3 was discovered to reach genome-wide significance.

An intronic variant in RCN3, 19:49541484:C:G (ENST00000270645 c.680-1069C>G; rs113886122), was associated with a higher exacerbation rate in subjects receiving dupilumab treatment (Table 1).

TABLE 1

| | | Effect | Samples |
|---|---|---|---|
| MAF | Pval | (LCI, UCI) | (RR\|RA\|AA) |
| 0.129 | 1.92E−08 | 0.822 | 649 |
| | | (0.535, 1.108) | 464\|140\|9 |

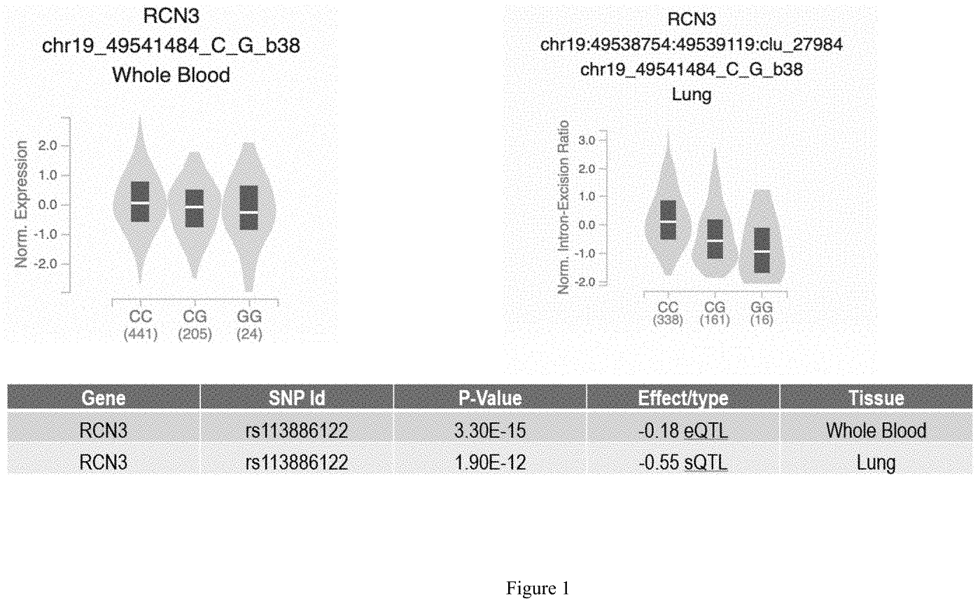

This variant is common with a minor allele frequency (MAF) close to 13%. Carriers of the G allele have a higher annualized exacerbation rate than non-carriers after dupilumab treatment. This variant was also associated with reduced RCN3 expression in Genotype-Tissue Expression (GTEX) when considering expression quantitative trait loci (eQTLs) and splicing quantitative trait loci (sQTLs) (FIG. 1). Interestingly, the intronic variant was found to be both an eQTL and sQTL for RCN3 in blood and lung tissues, respectively. For expression, the intronic variant was associated with reduced expression of RCN3 in whole blood. For splicing, the intronic variant was associated with significantly less effective splicing of known exons as compared to the reference assembly. Splicing was quantified using the intron excision phenotypes computed by LeafCutter (world wide web at “github.com/davidaknowles/leafcutter”).

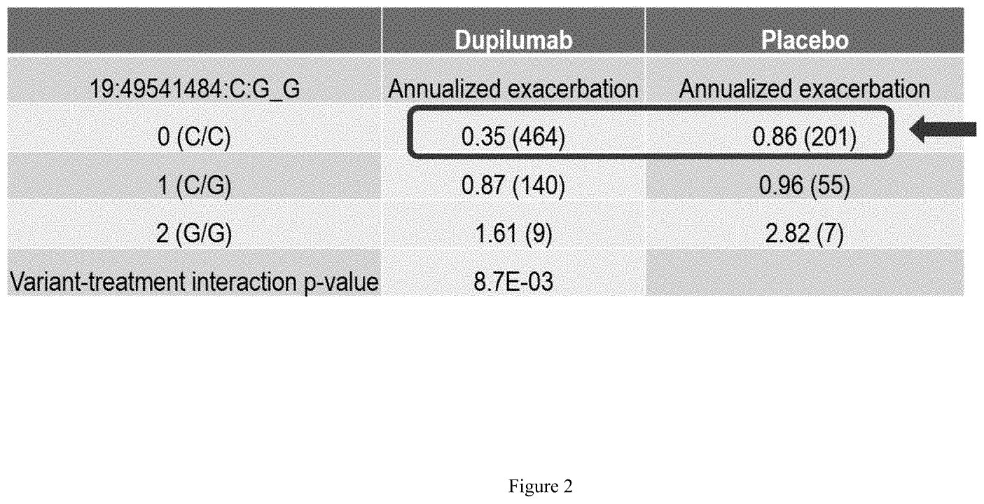

The annualized exacerbation rate or proportion of exacerbation for subjects carrying 0, 1 or two 19:49541484:C:G (rs113886122) alleles was determined (FIG. 2; median annualized exacerbation rate stratified by genotype in each arm). In the dupilumab-treated arm, carriers of the alternative G alleles had a higher exacerbation rate in a dosage-dependent manner. A significant reduction in the exacerbation rate for the non-carrier group (i.e., C/C consists of 76% of the enrolled European subjects) was observed. However, the carriers in dupilumab-treated arm still have a lower exacerbation rate than those in the placebo arm. The CC genotype has the lowest level of exacerbations in dupilumab treated patients. This indicates that dupilumab treatment is effective but to a lesser magnitude in those carrying one or two ALT alleles compared to the reference allele. This is consistent with the fact that this variant is associated with reduced RCN3 expression and knockouts of RCN3 in mice is associated with abnormal lung development or exacerbated lung fibrosis.

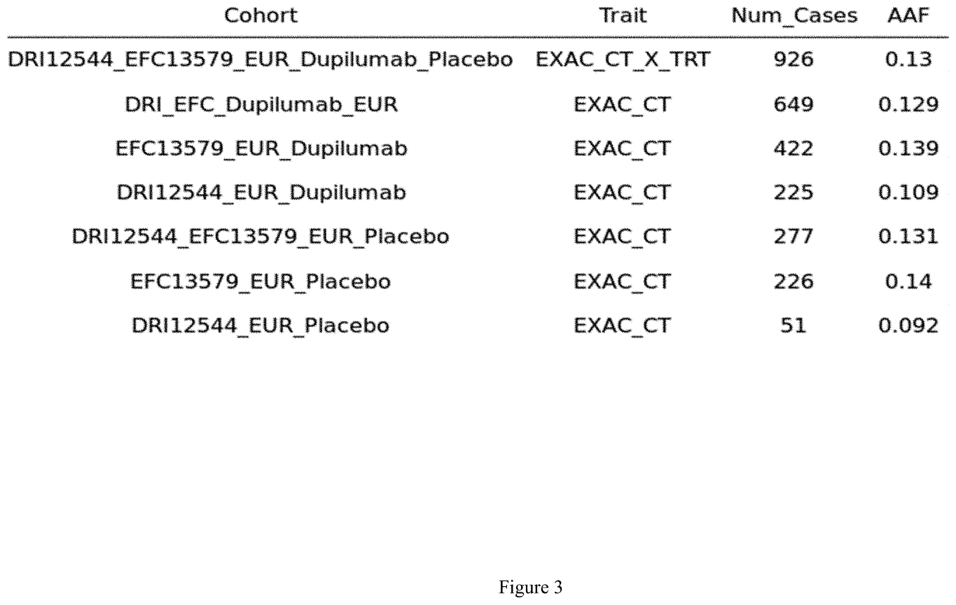

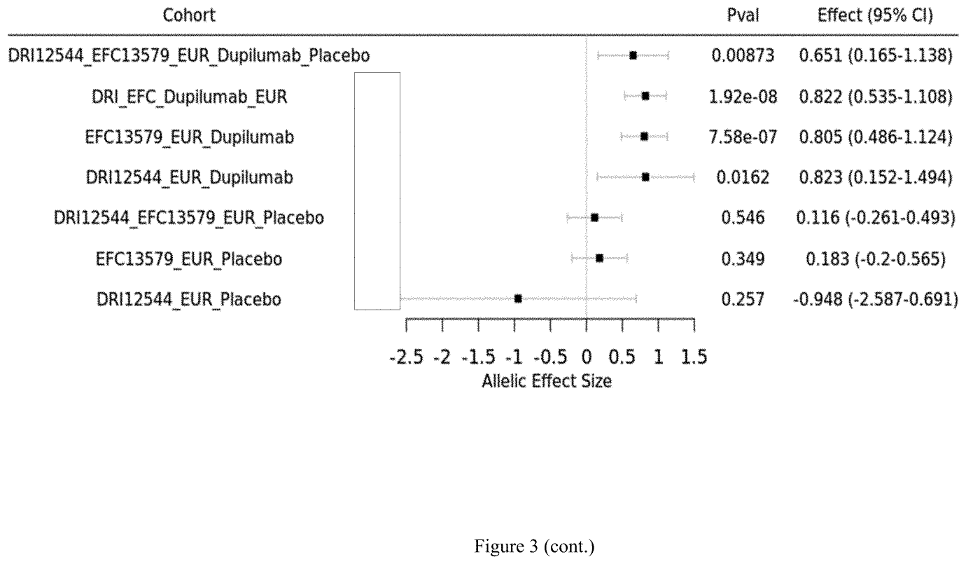

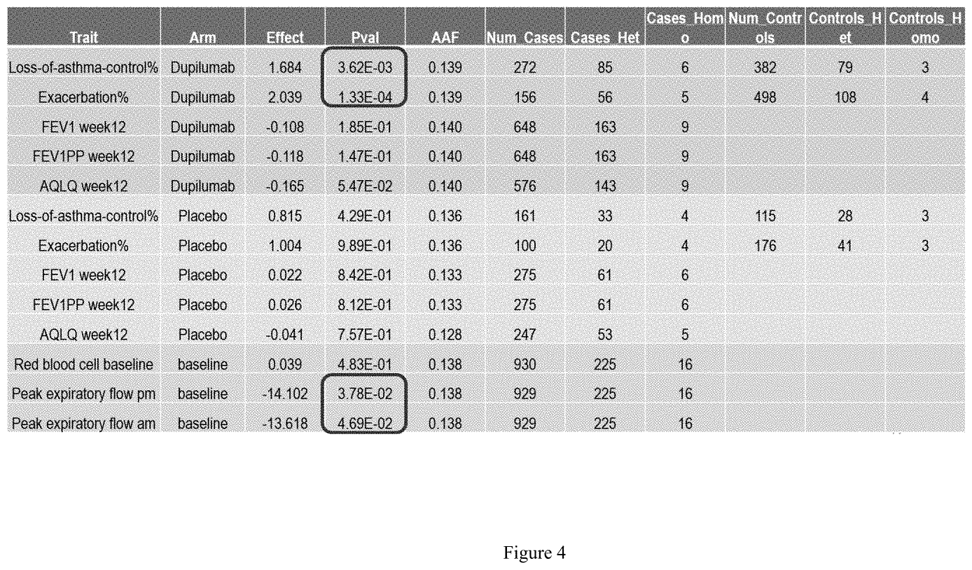

FIG. 3 also shows that the 19:49541484:C:G (rs113886122) variant is specifically associated with increased annualized exacerbation rates in dupilumab-treated asthma subjects. To further understand the effects of the 19:49541484:C:G (rs113886122) variant, other clinical measurements were considered (FIG. 4). In the analyses for the dupilumab arms (rows 1-5), significant association of the 19:49541484:C:G (rs113886122) allele was observed along with an increased proportion of subjects with a loss of asthma control or exacerbation. Rows 6-10 represent data for the placebo arm and no disease progress measurements were associated with the 19:49541484:C:G (rs113886122) variant. Rows 11-13 are baseline characteristics, where the 19:49541484:C:G (rs113886122) variant is associated with decreased lung function in asthma as measured by decreased peak expiratory flow.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of decreasing the asthma exacerbation rate in a subject having asthma, the method comprising:
determining whether the subject has a reticulocalbin-3 (RCN3) variant nucleic acid molecule by:
obtaining or having obtained a biological sample from the subject; and
performing or having performed a sequence analysis on the biological sample to determine if the subject has a genotype comprising the RCN3 variant nucleic acid molecule; and
administering or having administered an IL4R alpha antagonist and/or an IL13 blocking agent to a subject that is heterozygous or homozygous for the RCN3 variant nucleic acid molecule, thereby decreasing the asthma exacerbation rate in the subject.

2. The method according to claim 1, wherein the subject is heterozygous or homozygous for the RCN3 variant nucleic acid molecule, and the subject is administered the IL4R alpha antagonist and/or the IL13 blocking agent in an amount that is the same as or greater than a standard dosage amount and/or an RCN3 agonist.

3. The method according to claim 1, wherein the RCN3 variant nucleic acid molecule is a genomic nucleic acid molecule having a nucleotide sequence comprising a guanine at a position corresponding to position 13,482 according to SEQ ID NO:2.

4. The method according to claim 1, wherein the determining step is carried out in vitro.

5. The method according to claim 1, wherein the determining step comprises sequencing at least a portion of the nucleotide sequence of the RCN3 genomic nucleic acid molecule, or the complement thereof, in the biological sample, wherein the sequenced portion comprises a position corresponding to position 13,482 according to SEQ ID NO:2, or the complement thereof; wherein when the sequenced portion of the RCN3 genomic nucleic acid molecule in the biological sample comprises a guanine at a position corresponding to position 13,482 according to SEQ ID NO:2, or the complement thereof, then the RCN3 genomic nucleic acid molecule in the biological sample is an RCN3 variant genomic nucleic acid molecule.

6. The method according to claim 1, wherein the determining step comprises:
a) contacting the biological sample with a primer hybridizing to a portion of the nucleotide sequence of the RCN3 genomic nucleic acid molecule, or the complement thereof, that is proximate to a position corresponding to position 13,482 according to SEQ ID NO:2, or the complement thereof;
b) extending the primer at least through the position of the nucleotide sequence of the RCN3 genomic nucleic acid molecule, or the complement thereof, corresponding to position 13,482 according to SEQ ID NO:2, or the complement thereof; and
c) determining whether the extension product of the primer comprises a guanine at a position corresponding to position 13,482 according to SEQ ID NO:2, or the complement thereof.

7-10. (canceled)

11. The method according to claim 1, wherein the IL4R alpha antagonist and/or the IL13 blocking agent is dupilumab.

12. The method according to claim 1, wherein the RCN3 agonist is an RCN3 protein.

13. The method according to claim 1, the method further comprising administering or continuing to administer to the subject a therapeutic agent that treats or inhibits asthma exacerbation.

14. A method of treating a subject having asthma and who is undergoing or will be undergoing treatment with an IL4R alpha antagonist and/or an IL13 blocking agent, the method comprising:
determining whether the subject has a reticulocalbin-3 (RCN3) variant nucleic acid molecule by:
obtaining or having obtained a biological sample from the subject; and performing or having performed a sequence analysis on the biological sample to determine if the subject has a genotype comprising the RCN3 variant nucleic acid molecule; and
administering or continuing to administer the IL4R alpha antagonist and/or the IL13 blocking agent in a standard dosage amount to a subject that is RCN3 reference; and
administering or continuing to administer to a subject that is heterozygous or homozygous for the RCN3 variant nucleic acid molecule the IL4R alpha antagonist and/or the IL13 blocking agent in an amount that is the same as or greater than a standard dosage amount and/or an RCN3 agonist;
wherein the presence of a genotype in a subject that is homozygous for the RCN3 variant nucleic acid molecule indicates the subject has an increased risk of asthma exacerbation compared to a subject having a genotype that is heterozygous for the RCN3 variant nucleic acid molecule, and the presence of a genotype that is heterozygous for the RCN3 variant nucleic acid molecule indicates the subject has an increased risk of asthma exacerbation compared to a subject having a genotype that is RCN3 reference.

15. The method according to claim 14, wherein the subject is RCN3 reference, and the subject is administered or continued to be administered the IL4R alpha antagonist and/or the IL13 blocking agent in a standard dosage amount.

16. The method according to claim 14, wherein the subject is heterozygous or homozygous for the RCN3 variant nucleic acid molecule, and the subject is administered or continued to be administered the IL4R alpha antagonist and/or the IL13 blocking agent in an amount that is greater than a standard dosage amount.

17. The method according to claim 14, wherein the subject is heterozygous or homozygous for the RCN3 variant nucleic acid molecule, and the subject is administered or continued to be administered the IL4R alpha antagonist and/or the IL13 blocking agent in an amount that is the same as or greater than a standard dosage amount and/or an RCN3 agonist.

18. The method according to claim 14, wherein the RCN3 variant nucleic acid molecule is a genomic nucleic acid molecule having a nucleotide sequence comprising a guanine at a position corresponding to position 13,482 according to SEQ ID NO:2.

19. (canceled)

20. The method according to claim 14, wherein the determining step comprises sequencing at least a portion of the nucleotide sequence of the RCN3 genomic nucleic acid molecule, or the complement thereof, in the biological sample, wherein the sequenced portion comprises a position corresponding to position 13,482 according to SEQ ID NO:2, or the complement thereof; wherein when the sequenced portion of the RCN3 genomic nucleic acid molecule in the biological sample comprises a guanine at a position corresponding to position 13,482 according to SEQ ID NO:2, or the complement thereof, then the RCN3 genomic nucleic acid molecule in the biological sample is an RCN3 variant genomic nucleic acid molecule.

21. The method according to claim 14, wherein the determining step comprises:
a) contacting the biological sample with a primer hybridizing to a portion of the nucleotide sequence of the RCN3 genomic nucleic acid molecule, or the complement thereof, that is proximate to a position corresponding to position 13,482 according to SEQ ID NO:2, or the complement thereof;
b) extending the primer at least through the position of the nucleotide sequence of the RCN3 genomic nucleic acid molecule, or the complement thereof, corresponding to position 13,482 according to SEQ ID NO:2, or the complement thereof; and
c) determining whether the extension product of the primer comprises a guanine at a position corresponding to position 13,482 according to SEQ ID NO:2, or the complement thereof.

22-25. (canceled)

26. The method according to claim 14, wherein the IL4R alpha antagonist and/or the IL13 blocking agent is dupilumab.

27. The method according to claim 14, wherein the RCN3 agonist is an RCN3 protein.

28. The method according to claim 14, the method further comprising administering or continuing to administer to the subject a therapeutic agent that treats or inhibits asthma exacerbation.

29-41. (canceled)

42. A method of treating a subject having asthma or at risk of developing asthma and who is undergoing or will be undergoing treatment with an IL4R alpha antagonist and/or an IL13 blocking agent, the method comprising:
determining or having determined the subject's RCN3 gene expression score (RGES), wherein the RGES comprises a value determined from gene expression in a sample from the subject, and
when the subject's RGES is greater than a threshold RGES determined from a reference population of subjects without asthma, administering or continuing to administer to the subject the IL4R alpha antagonist and/or the 11,13 blocking agent in an amount that is the same as or greater than a standard dosage amount and/or an RCN3 agonist.

43-45. (canceled)
